(19) United States
(12) Patent Application Publication
(10) Pub. No.: US 2022/0002411 A1
(43) Pub. Date: Jan. 6, 2022

(54) METHODS FOR TREATING IMMUNE RELATED OCULAR DISORDERS

(71) Applicants: Huihui CHEN, Changsha, Hunan (CN); LEGEND MEDICAL-RIMONCI VISION TECHNOLOGY INCUBATION LIMITED, Central, Hong Kong (CN)

(72) Inventors: Huihui CHEN, Changsha, Hunan (CN); Guochun CHEN, Changsha, Hunan (CN)

(21) Appl. No.: 17/292,997

(22) PCT Filed: Nov. 10, 2019
(86) PCT No.: PCT/IB2019/059647

§ 371 Date: May 11, 2021

(51) Int. Cl. C07K 16/28; A61K 45/06; A61P 27/06

(52) U.S. Cl. CPC C07K 16/2818; C07K 16/2827; C07K 16/2878; A61P 27/06; C07K 16/2866; A61K 45/06; C07K 16/2875

(57) ABSTRACT

Disclosed is a method for treating ophthalmic inflammatory conditions in a subject by providing immune checkpoints inhibitors to the subject. Further disclosed are combinations of immune checkpoint inhibitors comprising enhanced therapeutic efficiency.

### FIELD OF DISCLOSURE

The disclosure presented herein provides methods for treating ophthalmic inflammatory conditions in a subject by providing immune checkpoints inhibitors to the subject. Further disclosed are combinations of immune checkpoint inhibitors comprising enhanced therapeutic efficiency.

### BACKGROUND

Several ophthalmic conditions are characterized by progressive degeneration of retinal ganglion cells (RGCs) and axons. In glaucoma, for example, elevated intraocular pressure (IOP), is thought to directly cause damage to neurons and the optic nerve. However, glaucomatous RGC and axon loss occur also in patients with normal IOP, or in patients whose IOP is effectively controlled by medical or surgical treatment. The most widely method for reducing IOP is trabeculectomy, in which a part of the eye's trabecular meshwork and adjacent structures are removed to allow drainage of aqueous humor from within the eye to underneath the conjunctiva where it is absorbed. Trabeculectomy has several drawbacks, as a high incidence of fluctuations in intraocular pressure, formation of cataracts, and postoperative complications with the bleb. Further, trabeculectomy is frequently ineffective.

Immune checkpoints are regulators of immune activation that play a key role in maintaining immune homeostasis and preventing autoimmunity. Uncontrolled immune responses can cause inflammatory tissue damage and autoimmune diseases. To prevent this, the magnitude of the immune response is regulated by a balance between stimulatory and inhibitory signals. This regulation is carried by immune checkpoints, which maintain self-tolerance and protect the host from tissue damage. In some clinical conditions, immune checkpoint signals are pathologically strengthened or weakened, jeopardizing the immune homeostasis. In cancer, for example, immune checkpoint mechanisms are often activated to suppress the nascent anti-tumor immune response.

Further, abundant experimental data indicates a role of immune checkpoints in autoimmune diseases. It is suggested, therefore, that inducing signaling through these immune checkpoints could switch off detrimental immune responses and drive the immune system back toward a state of tolerance after control has been lost in autoimmune disease.

Immune checkpoint proteins are divided into two major categories: inhibitory checkpoints, as PD1, CTLA-4 and VISTA, and stimulatory checkpoints, as CD28, CD86 and CD80. Several antibodies blocking immune checkpoints were developed in the last years, mainly for treating cancer and autoimmune disorders. These immune checkpoint inhibitors, as anti-PD1 molecules, showed significant anti-tumor efficacy. Further, recent studies indicate that administration of checkpoint inhibitors clear amyloid β deposits in mice models of Alzheimer, indicating a broader role of immune checkpoints in neuroinflammation.

It is clear that there remains a critical need for improved treatment of ophthalmic inflammatory conditions. The immune checkpoint inhibitors and methods disclosed herein have several features that make them advantageous over existing treatments for ophthalmic inflammatory conditions.

### SUMMARY OF THE DISCLOSURE

In one aspect, provided herein is a method for treating an ophthalmic inflammatory condition, the method comprising ocular administration of a composition comprising an immune checkpoint inhibitor comprising an antagonist of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR), wherein the administration treats the ophthalmic condition.

In a related aspect, the immune checkpoint inhibitor comprises a monoclonal antibody or a binding fragment thereof, a small molecule, or a peptide binding said immune checkpoint. In a related aspect, the said immune checkpoint inhibitor is selected from an anti-CD28, an anti-CD86, an anti-CD80, an anti-CD40, an anti-CD154, an anti-CD137, an anti-CD137L, an anti-CD27, an anti-CD70, an anti-CD122, an anti-CD48, an anti-CD278, an anti-CD275, an anti-CD357, an anti-CD279, an anti-CD134, an anti-CD255, or an anti-CD244 antibody, or adenosine.

In a related aspect, the immune checkpoint inhibitor is selected from varlilumab, nivolumab, belatacept, ASKP-1240, ISIS 19211, an IL-2/CD40L-expressing leukemia vaccine, 4SCAR19, 4SCAR70, abatacept, acalabrutinib ISIS9133, anti-thymocyte immunoglobulin, denileukin diftitox, AFTVac, GBR 830, BMS-663513, ipilimumab, ASKP-1240, IL-2/CD40L-expressing leukemia vaccine, ruplizumab, AMG 386, JTX-2011, TRX-518, alpha-D-mannose, adenosine, or IB-MECA, or any combination thereof. In a related aspect, the ophthalmic inflammatory condition comprises glaucoma, uveitis, age-related macular degeneration (AMD), diabetic retinopathy, proliferative vitreoretinopathy, acute optic nerve ischemia, keratitis, scleritis, optic neuritis, optic neuromyelitis, endophthalmitis, sputum cellulitis, retinitis pigmentosa, central retinal vein occlusion, central retinal artery occlusion, anterior ischemic optic neuropathy, thyroid associated ophthalmopathy, optic nerve maternal tumor, or choroidal melanoma, or a combination thereof.

In a related aspect, ocular administration comprises subconjunctival, intravitreal, retrobulbar, or intracameral administration, or any combination thereof. In a related aspect, the immune checkpoint inhibitor is administered at a dosage range of about 0.5-5 μg per administration. In a related aspect, the immune checkpoint inhibitor is administered once, or a number of times until achieving a desired therapeutic effect.

In a related aspect, treating comprises ameliorating a symptom comprising loss of peripheral vision, optic nerve cupping, thinning of the nerve fiber layer, severe unilateral eye pain, inflammation, cloudy vision, nausea and vomiting, red eye, swollen eye, eye enlargement, light sensitivity, tearing, or comprises reducing the concentration of immune cells or immune factors in a body fluid, or any combination thereof.

In some aspects, provided herein is a method for treating an ophthalmic inflammatory condition, the method comprising ocular administration of a composition comprising a combination of at least two immune checkpoint inhibitors selected from the group comprising an antagonist of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR), wherein said combination of immune checkpoints have a synergistic therapeutic effect, and wherein the administration treats the ophthalmic inflammatory condition.

In a related aspect, the immune checkpoint inhibitor comprises a monoclonal antibody or a binding fragment thereof, a small molecule, or a peptide binding said immune checkpoint. In a related aspect, the combination comprises an anti-CD28 and an anti-CD80 antibody; an anti-CD28 and an anti-CD86 antibody; an anti-CD28, an anti-CD80, and an anti-CD86 antibody; an anti-CD28 antibody and adenosine; an anti-CD28 antibody and an A2aR agonist; an anti-CD27 and anti-CD28 antibody, an anti-CD70 and an anti-CD85 antibody, an anti-CD27 and an anti-CD279 antibody, an anti-CD27 and an anti-CD273 antibody, an anti-CD27 and an anti-CD274 antibody, an anti-CD28 and an anti-CD279 antibody, an anti-CD28 and an anti-CD273 antibody, an anti-CD28 and an anti-CD274 antibody, an anti-CD80 and an anti-CD279 antibody, an anti-CD80 and an anti-CD273 antibody, an anti-CD80 and an anti-CD274 antibody, an anti-CD86 and an anti-CD279 antibody, an anti-CD86 and an anti-CD273 antibody, or an anti-CD86 and an anti-CD274 antibody.

In a related aspect, the immune checkpoint inhibitor is selected from varlilumab, nivolumab, belatacept, ASKP-1240, ISIS 19211, an IL-2/CD40L-expressing leukemia vaccine, 4SCAR19, 4SCAR70, abatacept, acalabrutinib ISIS9133, anti-thymocyte immunoglobulin, denileukin diftitox, AFTVac, GBR 830, BMS-663513, ipilimumab, ASKP-1240, IL-2/CD40L-expressing leukemia vaccine, ruplizumab, AMG 386, JTX-2011, TRX-518, alpha-D-mannose, adenosine, or IB-MECA, or any combination thereof.

In a related aspect, the ophthalmic inflammatory condition comprises glaucoma, uveitis, age-related macular degeneration (AMD), diabetic retinopathy, proliferative vitreoretinopathy, acute optic nerve ischemia, keratitis, scleritis, optic neuritis, optic neuromyelitis, endophthalmitis, sputum cellulitis, retinitis pigmentosa, central retinal vein occlusion, central retinal artery occlusion, anterior ischemic optic neuropathy, thyroid associated ophthalmopathy, optic nerve maternal tumor, or choroidal melanoma, or a combination thereof.

In a related aspect, the ocular administration comprises subconjunctival, intravitreal, retrobulbar, or intracameral administration, or any combination thereof. In a related aspect, the combination immune checkpoint inhibitor is administered at dosage of about range of about 0.5-5 μg. In a related aspect, the immune checkpoint inhibitor is administered once, or a number of times until achieving a desired therapeutic effect.

In a related aspect, treating comprises ameliorating a symptom comprising loss of peripheral vision, optic nerve cupping, thinning of the nerve fiber layer, severe unilateral eye pain, inflammation, cloudy vision, nausea and vomiting, red eye, swollen eye, eye enlargement, light sensitivity, tearing, or comprises reducing the concentration of immune cells or immune factors in a body fluid, or any combination thereof.

### BRIEF DESCRIPTION OF THE DRAWINGS

FIGS. 1A-1F show reduced ocular tissue damage in glaucoma mice treated with immune checkpoint inhibitors. Glaucoma was induced either by intraocular injection of polystyrene microparticles in C57BL/6J mice (FIGS. 1A-1C), or it developed spontaneously in DBA/2J transgenic mouse (FIGS. 1D-1F). Mice were treated with checkpoint inhibitors anti-CD28, anti-CD86, or anti-CD80, or with IgG for control. FIG. 1A shows decreased retinal ganglion cell (RGC) loss in glaucoma mice treated with a single checkpoint inhibitor. FIG. 1B shows decreased axonal loss in glaucoma mice treated with a single checkpoint inhibitor. FIG. 1C shows decreased RGC loss in glaucoma mice treated with a combination of 2 or 3 checkpoint inhibitors. DBA/2J transgenic mouse were treated by an intraocular injection of checkpoint inhibitors or IgG once a week from 3 month of age. FIG. 1D shows decreased RGC loss in mice 8 and 12 weeks after treatment with checkpoint inhibitors. FIGS. 1E and 1F show decreased CD4+/IFNγ+T cell ratio in eyes of mice treated with checkpoint inhibitors. *P<0.05, **P<0.01, ***P<0.001 by one-way ANOVA as compared to PBS injected group. Error bars: s.e.m. ###P<0.001, as compared to mice at 3 month of age. Error bars: s.e.m.

FIGS. 2A-2G show a reduced inflammatory response in glaucoma mice treated with CD28 or CD86 blocking antibodies. Anti-CD28 or IgG were injected into the vitreous cavity of high intraocular pressure mice. Peripheral blood was taken and IFNγ+, IL-4+, and IL-17+CD4+ cells were detected by flow cytometry. FIG. 2A shows a decrease of IFN+, IL4+, and IL17+CD4+ cells following intravitreal injection of anti-CD28 antibody compared to the IgG injected mice, indicating decreased Th1, Th2, and Th17 CD4 + T cells ratios. FIGS. 2B-2F show a decrease in CD4+ T cells and CD45RO+ memory T regulatory (mTreg) cells in glaucoma mice treated with an anti-CD28 or with anti-CD86 antibody. First, peripheral blood was isolated and labeled with an anti-CD4 antibody (FIG. 2B), CD4+ labeled T cells were then isolated and further labeled with anti-FOXP3 and anti-IL17 antibodies (FIG. 2C), then FOXP3+ regulatory T (Treg) cells were isolated and further labeled with anti-CD45RO and anti-CD45RA to assess the concentration of mTreg cells (FIG. 2D). FIG. 2E shows that both injection of anti-CD28 and of anti-CD86 antibodies reduced the number of CD4+ cells. FIG. 2F shows a decrease in CD45RO+ mTreg cells, but not in primitive CD45RA cells, following injection of anti-CD28 or of anti-CD86 antibodies. FIG. 2G shows Elispots confirming a significant decrease in T cell response in spleen 3 days (d), 1 week (w), and 4 weeks after injection of anti-CD28 antibody to glaucoma mice. *P<0.05, **P<0.01, ***P<0.001 by one-way ANOVA as compared to control group. Error bars: s.e.m.

FIGS. 3A and 3B show reduced ocular tissue damage in glaucoma mice treated with immune checkpoint inhibitors. Glaucoma was induced by intraocular injection of polystyrene microparticles in C57BL/6J mice. Mice were then treated with single immune checkpoint inhibitors anti-CD40, anti-CD154, anti-CD137, anti-CD137L, anti-CD27, anti-CD70, anti-CD122, anti-CD48, anti-CD278, anti-CD275, anti-CD357, anti-CD279, anti-CD134, anti-CD255, or anti-CD244 antibodies (FIG. 3A), with adenosine receptor agonists adenosine, A2aR agonist, or with combinations of checkpoint inhibitors adenosine and anti-CD28 antibody, A2aR and anti-CD28 antibody, adenosine and A2aR, or adenosine, A2aR and anti-CD28 antibody (FIG. 3B). FIG. 3A shows reduced RGC loss in mice treated with several immune checkpoint inhibitors. FIG. 3B shows reduced RGC loss in mice treated with adenosine receptor agonists, and enhancement of RGC protective effect by combinations of different checkpoint inhibitors. *P<0.05, **P<0.01, by one-way ANOVA as compared to control group. Error bars: s.e.m.

FIGS. 4A-4C show reduction of ocular tissue damage in mice with acute optic nerve ischemia following treatment with immune checkpoint inhibitors. Acute optic nerve ischemia was induced in C57BL/6J mice by the method of anterior chamber perfusion, and then treated with the immune checkpoint inhibitors anti-CD28, anti-CD86, anti-CD80, anti-CD27, or anti-CD70 antibody, with a combination of anti-CD27 and anti-CD28, or of anti-CD70 and anti-CD86 antibodies, or with IgG as control. FIG. 4A shows reduced RGC loss in mice treated with immune checkpoint inhibitors. FIG. 4B shows reduced axonal loss in mice treated with immune checkpoint inhibitors. FIG. 4C shows hematoxylin and eosin (HE) staining showed that the survival rate of a retinal ganglion cell layer in the antibody-injected group was significantly higher than that in the IgG-injected group, and no obvious glaucoma damage was observed in the thickness of the optic nerve fiber layer (C). *P<0.05, **P<0.01, by one-way ANOVA as compared to control group. Error bars: s.e.m.

FIGS. 5A-5C show reduction of ocular tissue damage in mice with uveitis following treatment with immune checkpoint inhibitors. Uveitis was induced in adult Lewis mice by immunization with HS-AgP35, and then treated with immune the checkpoint inhibitors anti-CD28, anti-CD86, and anti-CD80, anti-CD278, anti-CD70, anti-CD40, anti-CD154, or anti-CD122 antibodies, or with IgG as control. FIG. 5A shows reduced RGC loss in mice treated with immune checkpoint inhibitors. *P<0.05, **P<0.01, ***P<0.001 by one-way ANOVA as compared to control group. Error bars: s.e.m. FIG. 5B shows a decreased concentration of IFN-γ positive retinal cells in anti-CD28 antibody injected mice compared to control and IgG-injected mice. FIG. 5C shows the percentage of CD4+/IFN+ cells in retina of mice as indicated by flow cytometry analysis.

FIGS. 6A-6D show increased visual function in mice with diabetic retinopathy following treatment with immune checkpoint inhibitors. Diabetic retinopathy was induced in C57BL/6 mice by STZ injection. Mice were then treated with immune checkpoint inhibitors anti-CD27 and anti-CD28 antibodies twice a week for 3 months, or with IgG for control. FIG. 6A shows increased ERG a-waves in mice treated with immune checkpoint inhibitors. FIG. 6B shows increased ERG b-waves in mice treated with immune checkpoint inhibitors. FIG. 6C shows decreased concentration of CD4+/IFN-γ+ T cells in mice treated with checkpoint inhibitors. FIG. 6D shows growth of neovascular vessels (arrows). A large number of neovascular vessels are present in late-stage diabetic mice, but antibody-drug injection effectively reverses the pathological process). *P<0.05, **P<0.01, by one-way ANOVA as compared to control group. Error bars: s.e.m.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed disclosure.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

In the present disclosure the singular forms “a,” “an,” and “the” include the plural reference, and reference to a particular numerical value includes at least that particular value, unless the context clearly indicates otherwise. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

In some embodiments, the term “about”, refers to a deviance of between 0.0001-5% from the indicated number or range of numbers. In some embodiments, the term “about”, refers to a deviance of between 1-10% from the indicated number or range of numbers. In some embodiments, the term “about”, refers to a deviance of up to 25% from the indicated number or range of numbers.

This disclosure relates to methods for treating ophthalmic inflammatory conditions in a subject by providing immune checkpoints inhibitors to the subject. In some embodiments, disclosed herein are methods for treating an ophthalmic inflammatory condition in a subject, said method comprising ocular administration of a composition comprising an immune checkpoint inhibitor comprising an antagonist of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR) to the subject, thereby treating said ophthalmic inflammatory condition.

Further, the disclosure relates to combinations of immune checkpoint inhibitors comprising a synergistic therapeutic effect on ophthalmic conditions. In some embodiments, a combination of immune checkpoint inhibitors is selected from any combination of an inhibitor of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), and adenosine A3 receptor (A3aR).

In some embodiments, disclosed herein are methods for treating an ophthalmic inflammatory condition in a subject, said method comprising ocular administration of a composition comprising a combination of check point inhibitors or a combination of a check point inhibitor or check point inhibitors with an agonist of adenosine A1 receptor (A1aR), adenosine A2 receptor (A2aR), or adenosine A3 receptor (A3aR) to the subject, thereby treating said ophthalmic inflammatory condition. In some embodiments, the combination of checkpoint inhibitors comprises any combination of any antagonist of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR) to the subject.

### Immune Checkpoint Inhibitors

A skilled artisan would appreciate that the term “immune checkpoints” encompasses a group of proteins that regulates the extent and magnitude of immune responses, or inflammatory responses, by balancing between co-stimulatory and inhibitory signals. Activated T cells are primary mediators of immune effector functions and as such, they express multiple activating and inhibitory receptors such as lymphocyte-activation gene 3 (LAG-3), programmed cell death protein 1 (PD-1) and cytotoxic T-lymphocyte-associated protein 4 (CTLA-4). These immune checkpoint molecules protect the normal tissues from autoimmune damage, or when the immune system attacks pathogens or tumor cells.

In some embodiments, an immune checkpoint comprises a stimulatory checkpoint. In some embodiments, an immune checkpoint comprises an inhibitory checkpoint. In some embodiments, an immune checkpoint comprises only a fragment of an immune checkpoint. In some embodiments, an immune checkpoint comprises only the active epitope of an immune checkpoint.

In some embodiments, methods disclosed herein comprise use of an inhibitor of an immune check point molecule. In some embodiments, an inhibitor of an immune check point molecule comprises an antibody that blocks the activity of an immune check point molecule disclosed herein. In some embodiments, an inhibitor of an immune check point molecule comprises an antibody that reduces the activity of an immune check point molecule disclosed herein.

In some embodiments, an immune checkpoint comprises CD27. A skilled artisan would appreciate that CD27, also known as S152, S152. LPFS2, T14, TNFRSF7, and Tp55, is a receptor required for generation and long-term maintenance of T cell immunity, B-cell activation and immunoglobulin synthesis. CD27 comprises a number of isoforms, produced by alternatively spliced transcripts. All CD27 isoforms are encompassed by the term “CD27” as used herein. In some embodiments, human CD27 comprises an amino acid sequence comprising the NCBI accession number NP_001233. In some embodiments, it is encoded by the CD27 gene (NCBI GeneID: 939).

In some embodiments, an immune checkpoint comprises CD28. A skilled artisan would appreciate that CD28, also known as Tp44, is a protein essential for T-cell proliferation and survival, cytokine production, and T-helper type-2 development. CD28 comprises a number of isoforms, produced by alternatively spliced transcripts. All CD28 isoforms are encompassed by the term “CD28” as used herein. In some embodiments, human CD28 comprises an amino acid sequence comprising the NCBI accession number NP_006130. In some embodiments, it is encoded by the CD28 gene (NCBI GeneID: 940).

In some embodiments, an immune checkpoint comprises CD40. A skilled artisan would appreciate that CD40, also known as Bp50, CDW40, p50, tumor necrosis factor receptor superfamily member 5, TNFRSF5, is a receptor on antigen-presenting cells of the immune system and is essential for mediating a broad variety of immune and inflammatory responses including T cell-dependent immunoglobulin class switching, memory B cell development, and germinal center formation. CD40 comprises a number of isoforms, produced by alternatively spliced transcripts. All CD40 isoforms are encompassed by the term “CD40” as used herein. In some embodiments, human CD40 comprises an amino acid sequence comprising the NCBI accession number NP_001241. In some embodiments, it is encoded by the CD27 gene (NCBI GeneID: 958).

In some embodiments, an immune checkpoint comprises CD48. A skilled artisan would appreciate that CD48, also known as BCM1, BLAST, BLAST1, MEM-102, and SLAMF2, is found on the surface of lymphocytes and other immune cells, dendritic cells and endothelial cells, and participates in activation and differentiation pathways in these cells. CD48 comprises a number of isoforms, produced by alternatively spliced transcripts. All CD48 isoforms are encompassed by the term “CD48” as used herein. In some embodiments, human CD48 comprises an amino acid sequence comprising the NCBI accession number NP_001769. In some embodiments, it is encoded by the CD48 gene (NCBI GeneID: 962).

In some embodiments, an immune checkpoint comprises CD70. A skilled artisan would appreciate that CD70, also known as CD27L, LPFS3, CD27-L, CD27LG, TNFSF7, and TNLG8A, is a surface antigen on activated, but not on resting, T and B lymphocytes. It induces proliferation of costimulated T cells, enhances the generation of cytolytic T cells, and contributes to T cell activation. CD70 comprises two variants, produced by alternatively spliced transcripts. All CD70 isoforms are encompassed by the term “CD70” as used herein. In some embodiments, human CD70 comprises an amino acid sequence comprising the NCBI accession number NP_001243. In some embodiments, it is encoded by the CD70 gene (NCBI GeneID: 970).

In some embodiments, an immune checkpoint comprises CD80. A skilled artisan would appreciate that CD80, also known as B7, B7-1, B7.1, BB1, CD28LG, CD28LG1, and LAB7 is a membrane receptor activated by the binding of CD28 or CTLA-4, thus inducing T-cell proliferation and cytokine production. In some embodiments, human CD80 comprises an amino acid sequence comprising the NCBI accession number NP_005182. In some embodiments, it is encoded by the CD80 gene (NCBI GeneID: 941).

In some embodiments, an immune checkpoint comprises CD86. A skilled artisan would appreciate that CD86, also known as B7-2, B7.2, B70, CD28LG2, and LAB72, is the ligand for CD28 antigen and CTLA-4. Binding of CD86 with CD28 antigen is a costimulatory signal for activation of the T-cell. Binding of this protein with CTLA-4 negatively regulates T-cell activation and diminishes the immune response. CD86 comprises a number of isoforms, produced by alternatively spliced transcripts. All CD86 isoforms are encompassed by the term “CD86” as used herein. In some embodiments, human CD86 comprises an amino acid sequence comprising the NCBI accession number NP_787058. In some embodiments, it is encoded by the CD86 gene (NCBI GeneID: 942).

In some embodiments, an immune checkpoint inhibitor comprises CD122. A skilled artisan would appreciate that CD122, also known as IMD63, IL15RB, P70-75, and IL2RB, is involved in receptor-mediated endocytosis and transduction of mitogenic signals from interleukin 2. CD122 comprises a number of isoforms. All CD122 isoforms are encompassed by the term “CD122” as used herein. In some embodiments, human CD122 comprises an amino acid sequence comprising the NCBI accession number NP_001333151. In some embodiments, it is encoded by the CD122 gene (NCBI GeneID: 3560).

In some embodiments, an immune checkpoint comprises CD134. A skilled artisan would appreciate that CD134, also known as ACT35, TNFRRSF4, IMD16, OX40, and TXGP1L, is involved in the activation of inflammatory NF-kappaB. In some embodiments, human CD134 comprises an amino acid sequence comprising the NCBI accession number NP_003318. In some embodiments, it is encoded by the CD134 gene (NCBI GeneID: 7293).

In some embodiments, an immune checkpoint comprises CD137.A skilled artisan would appreciate that CD137, also known as IMD63, IL15RB, P70-75, and IL2RB, is involved in receptor-mediated endocytosis and transduction of mitogenic signals from interleukin 2. CD137 comprises a number of isoforms. All CD137 isoforms are encompassed by the term “CD137” as used herein. In some embodiments, human CD137 comprises an amino acid sequence comprising the NCBI accession number NP_001333151. In some embodiments, it is encoded by the CD137 gene (NCBI GeneID: 3560).

In some embodiments, an immune checkpoint comprises CD137L. A skilled artisan would appreciate that CD137L, also known as 4-1BB-L, TNFSF9, tumor necrosis factor ligand superfamily member 9, TNLG5A, is involved in the antigen presentation process and in the generation of cytotoxic T cells, and it has been shown to reactivate anergic T lymphocytes and promoting their proliferation. CD137L comprises a number of isoforms. All CD137L isoforms are encompassed by the term “CD137L” as used herein. In some embodiments, human CD137L comprises an amino acid sequence comprising the NCBI accession number NP_003802. In some embodiments, it is encoded by the CD137L gene (NCBI GeneID: 8744).

In some embodiments, an immune checkpoint comprises CD152. A skilled artisan would appreciate that CD152, also known as ALPS5, CELIAC3, CTLA-4, and GRD4, is involved in autoimmune disorders as insulin-dependent diabetes mellitus, Graves disease, Hashimoto thyroiditis, celiac disease, systemic lupus erythematosus, thyroid-associated orbitopathy. CD152 comprises a number of isoforms. All CD152 isoforms are encompassed by the term “CD152” as used herein. In some embodiments, human CD152 comprises an amino acid sequence comprising the NCBI accession number NP_001032720. In some embodiments, it is encoded by the CD152 gene (NCBI GeneID: 1493).

In some embodiments, an immune checkpoint comprises CD154. A skilled artisan would appreciate that CD154, also known as IGM, IMD3, TRAP, gp39, CD40L, HIGM1, T-BAM, TNFSF5, and hCD40L, regulates B cell function by engaging CD40 on the B cell surface. In some embodiments, human CD154 comprises an amino acid sequence comprising the NCBI accession number NP_000065. In some embodiments, it is encoded by the CD154 gene (NCBI GeneID: 959).

In some embodiments, an immune checkpoint comprises CD244. A skilled artisan would appreciate that CD244, also known as 2B4, NAIL, NKR2B4, Nmrk, and SLAMF4, modulates NK-cell cytolytic activity. CD244 comprises a number of isoforms. All CD244 isoforms are encompassed by the term “CD244” as used herein. In some embodiments, human CD244 comprises an amino acid sequence comprising the NCBI accession number NP_001160135. In some embodiments, it is encoded by the CD244 gene (NCBI GeneID: 51744).

In some embodiments, an immune checkpoint comprises CD252. A skilled artisan would appreciate that CD252, also known as P34, TNFSF4, OX4OL, TXGP1, CD134L, OX-40L, and TNLG2B, mediates adhesion of activated T cells to endothelial cells. CD252 comprises a number of isoforms. All CD252 isoforms are encompassed by the term “CD252” as used herein. In some embodiments, human CD252 comprises an amino acid sequence comprising the NCBI accession number NP_003317. In some embodiments, it is encoded by the CD252 gene (NCBI GeneID: 7292).

In some embodiments, an immune checkpoint comprises CD255. A skilled artisan would appreciate that CD255, also known as APO3L, DR3LG, TWEAK, TNLG4A, and TNFSF12, induces apoptosis, and promote proliferation and migration of endothelial cells, thus regulating angiogenesis. CD255 comprises a number of isoforms. All CD255 isoforms are encompassed by the term “CD255” as used herein. In some embodiments, human CD255 comprises an amino acid sequence comprising the NCBI accession number NP_003800. In some embodiments, it is encoded by the CD255 gene (NCBI GeneID: 8742).

In some embodiments, an immune checkpoint comprises CD273. A skilled artisan would appreciate that CD273, is also known as APO3L, DR3LG, TWEAK, TNLG4A, and TNFSF12. CD273 comprises a number of isoforms. All CD273 isoforms are encompassed by the term “CD273” as used herein. In some embodiments, human CD273 comprises an amino acid sequence comprising the NCBI accession number NP_079515. In some embodiments, it is encoded by the CD273 gene (NCBI GeneID: 803800).

In some embodiments, an immune checkpoint comprises CD274. A skilled artisan would appreciate that CD274, also known as B7-H, B7H1, PD-L1, PDCD1L1, PDCD1LG1, PDL1, and hPD-L1, inhibits T-cell activation and cytokine production, thus preventing autoimmunity by maintaining homeostasis of the immune response. CD274 comprises a number of isoforms. All CD274 isoforms are encompassed by the term “CD274” as used herein. In some embodiments, human CD274 comprises an amino acid sequence comprising the NCBI accession number NP_001254635. In some embodiments, it is encoded by the CD274 gene (NCBI GeneID: 29126).

In some embodiments, an immune checkpoint comprises CD275. A skilled artisan would appreciate that CD275, is also known as B7h, B7H2, GL50, B7-H2, B7RP1, ICOSLG, ICOSL, LICOS, and B7RP-1. In some embodiments, human CD275 comprises an amino acid sequence comprising the NCBI accession number NP_001352688. In some embodiments, it is encoded by the CD275 gene (NCBI GeneID: 23308).

In some embodiments, an immune checkpoint comprises CD278. A skilled artisan would appreciate that CD278, also known as ICOS, AILIM, and CVID1, plays an important role in cell-cell signaling, immune responses, and regulation of cell proliferation. In some embodiments, human CD278 comprises an amino acid sequence comprising the NCBI accession number NP_036224. In some embodiments, it is encoded by the CD278 gene (NCBI GeneID: 29851).

In some embodiments, an immune checkpoint comprises CD357. A skilled artisan would appreciate that CD357, also known as AITR, GITR, TNFRSF18, GITR-D, and ENERGEN, plays a key role in dominant immunological self-tolerance. CD357 comprises a number of isoforms. All CD357 isoforms are encompassed by the term “CD357” as used herein. In some embodiments, human CD357 comprises an amino acid sequence comprising the NCBI accession number NP_004186. In some embodiments, it is encoded by the CD357 gene (NCBI GeneID: 29126).

In some embodiments, an immune checkpoint comprises CD357. A skilled artisan would appreciate that CD357, also known as AITR, GITR, TNFRSF18, GITR-D, and ENERGEN, plays a key role in dominant immunological self-tolerance. CD357 comprises a number of isoforms. All CD357 isoforms are encompassed by the term “CD357” as used herein. In some embodiments, human CD357 comprises an amino acid sequence comprising the NCBI accession number NP_004186. In some embodiments, it is encoded by the CD357 gene (NCBI GeneID: 29126).

In some embodiments, an immune checkpoint comprises GITRL. A skilled artisan would appreciate that GITRL, also known as AITRL, TL6, TNLG2A, hGITRL, and TNFSF18 modulates T lymphocyte survival in peripheral tissues. In some embodiments, human GITRL comprises an amino acid sequence comprising the NCBI accession number NP_005083. In some embodiments, it is encoded by the GITRL gene (NCBI GeneID: 8995).

In some embodiments, an immune checkpoint comprises BTN2A1. A skilled artisan would appreciate that BTN2A1, also known as BK14H9.1, BT2.1, BTF1, BTN2.1, and DJ3E1.1, is an integral plasma membrane protein involved in lipid, fatty-acid, and sterol metabolism. BTN2A1 comprises a number of isoforms. All BTN2A1 isoforms are encompassed by the term “BTN2A1” as used herein. In some embodiments, human BTN2A1 comprises an amino acid sequence comprising the NCBI accession number NP_001184162. In some embodiments, it is encoded by the BTN2A1 gene (NCBI GeneID: 11120).

In some embodiments, an immune checkpoint comprises DC-SIGN. A skilled artisan would appreciate that DC-SIGN, also known as CD209, CDSIGN, CLEC4L, and DC-SIGN1, is involved in the innate immune system and recognizes numerous evolutionarily divergent pathogens ranging from parasites to viruses with a large impact on public health. DC-SIGN comprises a number of isoforms. All DC-SIGN isoforms are encompassed by the term “DC-SIGN” as used herein. In some embodiments, human DC-SIGN comprises an amino acid sequence comprising the NCBI accession number NP_066978. In some embodiments, it is encoded by the DC-SIGN gene (NCBI GeneID: 30835).

In some embodiments, an immune checkpoint comprises TL1A. A skilled artisan would appreciate that TL1A, also known as TL1, TNFSF15, VEGI, TNLG1B, and VEGI192A, activates inflammatory NF-kappaB and MAP kinases, and acts as an autocrine factor to induce apoptosis in endothelial cells. TL1A comprises two variants. All TL1A isoforms are encompassed by the term “TL1A” as used herein. In some embodiments, human TL1A comprises an amino acid sequence comprising the NCBI accession number NP_001184162. In some embodiments, it is encoded by the TL1A gene (NCBI GeneID: 11120).

In some embodiments, an immune checkpoint comprises DR3. A skilled artisan would appreciate that DR3, also known as TNFRSF25, APO-3, DDR3, DR3, LARD, TNFRSF12, TR3, TRAMP, WSL-1, WSL-LR, GEF720, and PLEKHG5, stimulates NF-kappa B activity and regulate cell apoptosis. DR3 comprises several isoforms produced by alternative splicing. All DR3 isoforms are encompassed by the term “DR3” as used herein. In some embodiments, human DR3 comprises an amino acid sequence comprising the NCBI accession number NP_683866. In some embodiments, it is encoded by the DR3 gene (NCBI GeneID: 8718).

In some embodiments, an immune checkpoint comprises A1aR. A skilled artisan would appreciate that A1aR, is also known as Adora1, Ri, A1R, AA1R, ARA1, AI848715, and BB176431. A1aR comprises a number of alternatively spliced isoforms. All A1aR isoforms are encompassed by the term “TL1A” as used herein. In some embodiments, human A1aR comprises an amino acid sequence comprising the NCBI accession number NP_001041695. In some embodiments, it is encoded by the A1aR gene (NCBI GeneID: 134).

In some embodiments, an immune checkpoint comprises A2aR. A skilled artisan would appreciate that A2aR, also known as ADORA2A, RDC8, ADORA2, is implicated in pathophysiological conditions such as inflammatory diseases and neurodegenerative disorders. A2aR comprises several isoforms produced by alternative splicing. All A2aR isoforms are encompassed by the term “A2aR” as used herein. In some embodiments, human A2aR comprises an amino acid sequence comprising the NCBI accession number NP_001265429. In some embodiments, it is encoded by the A2aR gene (NCBI GeneID: 135).

In some embodiments, an immune checkpoint comprises A3aR. A skilled artisan would appreciate that A3aR, also known as ADORA3 and adenosine A3 receptor, is involved in the inhibition of neutrophil degranulation in neutrophil-mediated tissue injury. A3aR comprises several isoforms produced by alternative splicing. All A3aR isoforms are encompassed by the term “A2aR” as used herein. In some embodiments, human A3aR comprises an amino acid sequence comprising the NCBI accession number NP_000668. In some embodiments, it is encoded by the A3aR gene (NCBI GeneID: 140).

As immune checkpoint mechanisms are often activated to suppress anti-tumor immune response, several checkpoint inhibitors, including anti-checkpoint antibodies, are commercially available or being tested in clinical trials for cancer as well as other immune-related disorders.

In some embodiments, an immune checkpoint inhibitor comprises an antibody reactive with the immune checkpoint. In some embodiments, an immune checkpoint inhibitor comprises an antibody that binds an immune checkpoint described herein. In some embodiments, an immune checkpoint inhibitor comprises an antibody that inactivates an immune checkpoint, as described herein.

As used herein, the term “antibody” or “immunoglobulin” is intended to encompass both polyclonal, monoclonal antibodies, and binding fragments thereof. The term “antibody” is also intended to encompass mixtures of more than one antibody reactive with the antigen (e.g., a cocktail of different types of monoclonal antibodies reactive with the antigen). In some embodiments, the term “antibody” encompasses whole antibodies, biologically functional fragments thereof, single-chain antibodies, and genetically altered antibodies such as chimeric antibodies comprising portions from more than one species, bifunctional antibodies, antibody conjugates, humanized and human antibodies.

Biologically functional antibody fragments or “binding fragments”, which can also be used, are those peptide fragments derived from an antibody that are sufficient for binding to the antigen. “Antibody” as used herein is meant to include the entire antibody as well as any antibody fragments capable of binding the epitope, antigen or antigenic fragment of interest.

In some embodiments, the term “antibody” as used herein comprises a fragment secondary antibody. In some embodiments, the term “antibody” as used herein comprises a F(ab′)2 fragment. In some embodiments, the term “antibody” as used herein comprises a Fab′ fragment. In some embodiments, the term “antibody” as used herein comprises a Fab fragment. In some embodiments, the term “antibody” as used herein comprises a scFv fragment. In some embodiments, the term “antibody” as used herein comprises a bivalent scFv fragment. In some embodiments, the term “antibody” as used herein comprises a trivalent scFv fragment. In some embodiments, the term “antibody” as used herein comprises a Fv fragment.

In some embodiments, an immune checkpoint inhibitor comprises a monoclonal antibody. In some embodiments, an immune checkpoint inhibitor comprises a polyclonal antibody. In some embodiments, the antibody comprises a chimeric antibody.

In some embodiments, the antibody comprises a humanized antibody. Generally, a humanized antibody comprises one or more amino acid residues introduced into it from a source that is non-human. Humanization can be performed, for example, using methods described in the art, by substituting at least a portions of the non-human antibody with the corresponding portions of a human antibody. In some embodiments, activity of the immune check point is reduced or inhibited by a checkpoint inhibitor. In some embodiments, an antibody immune check point inhibitor or active fragment thereof, reduces or inhibits the activity of the immune checkpoint molecule. In some embodiments, an antibody immune check point inhibitor or active fragment thereof, reduces the activity of the immune checkpoint molecule. In some embodiments, an antibody immune check point inhibitor or active fragment thereof, inhibits the activity of the immune checkpoint molecule.

In some embodiments, an antibody immune check point inhibitor or active fragment thereof, reduces the activity of a check point molecule by about 5%-100%. In some embodiments, an antibody immune check point inhibitor or active fragment thereof, reduces the activity of a check point molecule by about 5%-10%. In some embodiments, an antibody immune check point inhibitor or active fragment thereof, reduces the activity of a check point molecule by about 10%-20%. In some embodiments, an antibody immune check point inhibitor or active fragment thereof, reduces the activity of a check point molecule by about 30-40%. In some embodiments, an antibody immune check point inhibitor or active fragment thereof, reduces the activity of a check point molecule by about 40-50%.

In some embodiments, an antibody immune check point inhibitor or active fragment thereof, reduces the activity of a check point molecule by about 50-60%. In some embodiments, an antibody immune check point inhibitor or active fragment thereof, reduces the activity of a check point molecule by about 60-70%. In some embodiments, an antibody immune check point inhibitor or active fragment thereof, reduces the activity of a check point molecule by about 70-80%. In some embodiments, an antibody immune check point inhibitor or active fragment thereof, reduces the activity of a check point molecule by about 80-90%. In some embodiments, an antibody immune check point inhibitor or active fragment thereof, reduces the activity of a check point molecule by about 90-99%. In some embodiments, an antibody immune check point inhibitor or active fragment thereof, reduces the activity of a check point molecule by about 90-100%.

In some embodiments, an antibody immune check point inhibitor or active fragment thereof, reduces the activity of a check point molecule by about 0-70%. In some embodiments, an antibody immune check point inhibitor or active fragment thereof, reduces the activity of a check point molecule by about 10-60%. In some embodiments, an antibody immune check point inhibitor or active fragment thereof, reduces the activity of a check point molecule by about 20-50%.

In some embodiments, an antibody immune check point inhibitor or active fragment thereof, inhibits the activity of a check point molecule by about 5%-100%. In some embodiments, an antibody immune check point inhibitor or active fragment thereof, inhibits the activity of a check point molecule by about 5%-10%. In some embodiments, an antibody immune check point inhibitor or active fragment thereof, inhibits the activity of a check point molecule by about 10%-20%. In some embodiments, an antibody immune check point inhibitor or active fragment thereof, inhibits the activity of a check point molecule by about 30-40%. In some embodiments, an antibody immune check point inhibitor or active fragment thereof, inhibits the activity of a check point molecule by about 40-50%. In some embodiments, an antibody immune check point inhibitor or active fragment thereof, inhibits the activity of a check point molecule by about 50-60%. In some embodiments, an antibody immune check point inhibitor or active fragment thereof, inhibits the activity of a check point molecule by about 60-70%. In some embodiments, an antibody immune check point inhibitor or active fragment thereof, inhibits the activity of a check point molecule by about 70-80%. In some embodiments, an antibody immune check point inhibitor or active fragment thereof, inhibits the activity of a check point molecule by about 80-90%. In some embodiments, an antibody immune check point inhibitor or active fragment thereof, inhibits the activity of a check point molecule by about 90-99%. In some embodiments, an antibody immune check point inhibitor or active fragment thereof, inhibits the activity of a check point molecule by about 90-100%.

In some embodiments, an immune checkpoint inhibitor comprises a small molecule. In some embodiments, an immune checkpoint inhibitor comprises a peptide. In some embodiments, an immune checkpoint inhibitor comprises a polypeptide or a protein. In some embodiments, an immune checkpoint inhibitor comprises a naturally occurring checkpoint inhibitor, or a fragment thereof. In some embodiments, an immune checkpoint inhibitor comprises a synthetic checkpoint inhibitor.

In some embodiments, a checkpoint inhibitor comprises a molecule that down-regulates or blocks the expression of the immune checkpoint. In some embodiments, a checkpoint inhibitor comprises a molecule that down-regulates the expression of the immune checkpoint. In some embodiments, a checkpoint inhibitor comprises a molecule that blocks the expression of the immune checkpoint. In some embodiments, the checkpoint inhibitor that down-regulates or blocks the expression of the immune checkpoint comprises an antibody, or active fragment thereof, a small molecule, an siRNA, an antisense RNA, an miRNA, a shRNA, a peptide, a polypeptide, or a protein, as described herein.

In some embodiments, the expression of the immune check point is down regulated by about 5%-100%. In some embodiments, the expression of the immune check point is down regulated by about 10%-20%. In some embodiments, the expression of the immune check point is down regulated by about 20-30%. In some embodiments, the e In some embodiments, the expression of the immune check point is down regulated by about 5%-100%. In some embodiments, the expression of the immune check point is down regulated by about 10%-20%. In some embodiments, the expression of the immune check point is down regulated by about 20-30%. In some embodiments, the expression of the immune check point is down regulated by about 30-40%. In some embodiments, the expression of the immune check point is down regulated by about 40-50%. In some embodiments, the expression of the immune check point is down regulated by about 50-60%. In some embodiments, the expression of the immune check point is down regulated by about 60-70%. In some embodiments, the expression of the immune check point is down regulated by about 70-80%. In some embodiments, the expression of the immune check point is down regulated by about 80-90%. In some embodiments, the expression of the immune check point is down regulated by about 90-99%. In some embodiments, the expression of the immune check point is down regulated by about 90-100%.

In some embodiments, expression of the immune check point is blocked by about 30-40%. In some embodiments, the expression of the immune check point is blocked by about 40-50%. In some embodiments, the expression of the immune check point is blocked by about 50-60%. In some embodiments, the expression of the immune check point is blocked by about 60-70%. In some embodiments, the expression of the immune check point is blocked by about 70-80%. In some embodiments, the expression of the immune check point is blocked by about 80-90%. In some embodiments, the expression of the immune check point is blocked by about 90-99%. In some embodiments, the expression of the immune check point is blocked by about 90-100%.

In some embodiments, expression of the immune check point is blocked by about 0-70% In some embodiments, expression of the immune check point is blocked by about 10-60% In some embodiments, expression of the immune check point is blocked by about 20-50%.

In some embodiments, a checkpoint inhibitor comprises a siRNA, an antisense, a miRNA, or a shRNA. In some embodiments, a siRNA, an antisense RNA, a miRNA, or a shRNA is complementary to a fragment of a checkpoint inhibitor mRNA. In some embodiments, a siRNA, an antisense, a miRNA, or a shRNA binds a fragment of a checkpoint inhibitor mRNA.

In some embodiments, a siRNA, an antisense, a miRNA, or a shRNA down-regulate the expression of the immune checkpoint by about 0 to 10%. In some embodiments, a siRNA, an antisense, a miRNA, or a shRNA down-regulate the expression of the immune checkpoint by about 10% to 20%. In some embodiments, a siRNA, an antisense, a miRNA, or a shRNA down-regulate the expression of the immune checkpoint by about 20% to 30%. In some embodiments, a siRNA, an antisense, a miRNA, or a shRNA down-regulate the expression of the immune checkpoint by about 30% to 40%.

In some embodiments, a siRNA, an antisense, a miRNA, or a shRNA down-regulate the expression of the immune checkpoint by about 40% to 50%. In some embodiments, a siRNA, an antisense, a miRNA, or a shRNA down-regulate the expression of the immune checkpoint by about 50% to 60%. In some embodiments, a siRNA, an antisense, a miRNA, or a shRNA down-regulate the expression of the immune checkpoint by about 60% to 70%. In some embodiments, a siRNA, an antisense, a miRNA, or a shRNA down-regulate the expression of the immune checkpoint by about 80% to 90%. In some embodiments, a siRNA, an antisense, a miRNA, or a shRNA down-regulate the expression of the immune checkpoint by about 90% to 100%.

In some embodiments, the terms “inhibitor”, “antagonist”, and “blocker” are used herein interchangeably having all the same qualities and meanings.

In some embodiments, a CD27 inhibitor comprises an anti-CD27 antibody. In some embodiments, a CD27 inhibitor comprises Varlilumab (CDX-1127). In some embodiments, a CD28 inhibitor comprises an anti-CD28 antibody. In some embodiments, a CD28 inhibitor comprises Belatacept. In some embodiments, a CD40 inhibitor comprises an anti-CD40 antibody. In some embodiments, a CD40 inhibitor comprises ASKP-1240, ISIS 19211, or IL-2/CD40L-expressing leukemia vaccine. In some embodiments, a CD40 inhibitor comprises 4SCAR19 or 4SCAR70.

In some embodiments, a CD48 inhibitor comprises an anti-CD48 antibody. In some embodiments, a CD70 inhibitor comprises an anti-CD70 antibody. In some embodiments, a CD80 inhibitor comprises an anti-CD80 antibody. In some embodiments, a CD80 inhibitor comprises Abatacept, Galiximab, Belatacept, Macrocycle derivative 6, ISIS 13805, or Durvalumab.

In some embodiments, a CD86 inhibitor comprises an anti-CD86 antibody. In some embodiments, a CD86 inhibitor comprises Abatacept, Acalabrutinib, Belatacept, ISIS 9133, or anti-thymocyte immunoglobulin. In some embodiments, a CD122 inhibitor comprises an anti-CD122 antibody. In some embodiments, a CD122 inhibitor comprises denileukin diftitox or AFTVac. In some embodiments, a 134 inhibitor comprises GBR 830.

In some embodiments, a CD134 inhibitor comprises an anti-CD134 antibody. In some embodiments, a CD137 inhibitor comprises an anti-CD137 antibody. In some embodiments, a 137 inhibitor comprises BMS-663513.

In some embodiments, a CD137L inhibitor comprises an anti-137L antibody. In some embodiments, a CD152 inhibitor comprises an anti-CD152 antibody. In some embodiments, a CD152 inhibitor comprises Ipilimumab. In some embodiments, a CD154 inhibitor comprises an anti-CD154 antibody. In some embodiments, a CD154 inhibitor comprises ASKP-1240, IL-2/CD40L-expressing leukemia vaccine, or Ruplizumab.

In some embodiments, a CD154 inhibitor comprises an anti-CD154 antibody. In some embodiments, a CD244 inhibitor comprises an anti-CD244 antibody. In some embodiments, a CD252 inhibitor comprises an anti-CD252 antibody. In some embodiments, a CD252 inhibitor comprises AMG 386. In some embodiments, a CD255 inhibitor comprises an anti-CD255 antibody. In some embodiments, a CD273 inhibitor comprises an anti-CD273 antibody.

In some embodiments, a CD274 inhibitor comprises an anti-CD274 antibody. In some embodiments, a CD275 inhibitor comprises an anti-CD275 antibody. In some embodiments, a CD275 inhibitor comprises AMG 557. In some embodiments, a CD278 inhibitor comprises an anti-CD278 antibody. In some embodiments, a CD278 inhibitor comprises JTX-2011. In some embodiments, a CD357 inhibitor comprises an anti-CD357 antibody. In some embodiments, a CD357 inhibitor comprises TRX-518.

In some embodiments, a GITRL inhibitor comprises an anti-GITRL antibody. In some embodiments, a BTN2A1 inhibitor comprises an anti-BTN2A1 antibody. In some embodiments, a DC-SIGN inhibitor comprises an anti-DC-SIGN antibody. In some embodiments, a DC-SIGN inhibitor comprises Alpha-D-Mannose.

In some embodiments, a TL1A inhibitor comprises an anti-TL1A antibody. In some embodiments, a DR3 inhibitor comprises an anti-DR3 antibody. In some embodiments, an A1aR, A2aR, or A3aR inhibitor comprises adenosine. In some embodiments, an A3aR inhibitor comprises IB-MECA.

In some embodiments, disclosed herein are methods of treating an ophthalmic inflammatory condition in a subject, said method comprising ocular administration of a composition comprising a combination of at least two immune checkpoint inhibitors to the subject, wherein the immune checkpoint inhibitors are selected from the group comprising an antagonist of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR).

In some embodiments, a composition comprising a combination of immune checkpoint inhibitors comprises any two of the group comprising an antagonist of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR). In some embodiments, a composition comprising a combination of immune checkpoint inhibitors comprises any three of the group comprising an antagonist of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR).

In some embodiments, a composition comprising a combination of immune checkpoint inhibitors comprises any four or more of the group comprising an antagonist of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR).

In some embodiments, the composition comprises a combination of a CD28 and a CD80 inhibitor. In some embodiments, the composition comprises a combination of a CD28 and a CD86 inhibitor. In some embodiments, the composition comprises a combination of a CD28, a CD80, and a CD86 inhibitor. In some embodiments, the composition comprises a combination of a CD28 inhibitor and adenosine. In some embodiments, the composition comprises a combination of a CD28 inhibitor and an A2aR agonist. In some embodiments, the composition comprises a combination of a CD28 inhibitor, adenosine, and an A2aR agonist. In some embodiments, the composition comprises a combination of a CD27 and a CD28 inhibitor. In some embodiments, the composition comprises a combination of a CD70 and a CD86 inhibitor.

In some embodiments, the composition comprises a combination of a CD27 and a CD279 inhibitor. In some embodiments, the composition comprises a combination of a CD27 and a CD273 inhibitor. In some embodiments, the composition comprises a combination of a CD27 and a CD274 inhibitor. In some embodiments, the composition comprises a combination of a CD28 and a CD279 inhibitor. In some embodiments, the composition comprises a combination of a CD28 and a CD273 inhibitor. In some embodiments, the composition comprises a combination of a CD28 and a CD274 inhibitor.

In some embodiments, the composition comprises a combination of a CD80 and a CD279 inhibitor. In some embodiments, the composition comprises a combination of a CD80 and a CD273 inhibitor. In some embodiments, the composition comprises a combination of a CD80 and a CD274 inhibitor. In some embodiments, the composition comprises a combination of a CD86 and a CD279 inhibitor. In some embodiments, the composition comprises a combination of a CD86 and a CD273 inhibitor. In some embodiments, the composition comprises a combination of a CD86 and a CD274 inhibitor.

In some embodiments, the combination comprises an anti-CD28 and an anti-CD80 antibody. In some embodiments, the combination comprises an anti-CD28 and an anti-CD86 antibody. In some embodiments, the combination comprises an anti-CD28, an anti-CD80, and an anti-CD86 antibody. In some embodiments, the combination comprises an anti-CD28 antibody and adenosine. In some embodiments, the combination comprises an anti-CD28 antibody and an A2aR agonist. In some embodiments, the combination comprises an anti-CD27 and anti-CD28 antibody. In some embodiments, the combination comprises an anti-CD70 and an anti-CD85 antibody.

In some embodiments, the combination comprises an anti-CD27 and an anti-CD279 inhibitor. In some embodiments, the combination comprises an anti-CD27 and an anti-CD273 inhibitor. In some embodiments, the combination comprises an anti-CD27 and an anti-CD274 inhibitor. In some embodiments, the combination comprises an anti-CD28 and an anti-CD279 inhibitor. In some embodiments, the combination comprises an anti-CD28 and an anti-CD273 inhibitor. In some embodiments, the combination comprises a combination of a CD28 and an anti-CD274 inhibitor.

In some embodiments, the combination comprises an anti-CD80 and an anti-CD279 inhibitor. In some embodiments, the combination comprises an anti-CD80 and an anti-CD273 inhibitor. In some embodiments, the combination comprises an anti-CD80 and an anti-CD274 inhibitor. In some embodiments, the combination comprises an anti-CD86 and an anti-CD279 inhibitor. In some embodiments, the combination comprises an anti-CD86 and an anti-CD273 inhibitor. In some embodiments, the combination comprises an anti-CD86 and an anti-CD274 inhibitor.

In some embodiments, disclosed herein is a method for treating an ophthalmic inflammatory condition in a subject, the method comprising:
(a) measuring T cell concentration and/or activity in a body fluid of said subject, and
(b) if the T cell concentration is above a pre-determined threshold, providing to said subject a composition comprising an immune checkpoint inhibitor.

In some embodiments, disclosed herein is a method for treating an ophthalmic inflammatory condition in a subject, the method comprising:
(a) measuring T cell concentration and/or activity in the retina of said subject, and
(b) if the T cell concentration is above a pre-determined threshold, providing to said subject a composition comprising an immune checkpoint inhibitor.

In some embodiments, a body fluid comprises blood, blood plasma, serum, aqueous humour, vitreous body, interstitial fluid, or lymph, or any combination thereof. In some embodiments, the activity of T cells can be determined by detecting characteristic biomarkers or cytokine spectral responses, including the signature secretory factor profiles of T cell subsets, as IFN-γ, IL-17, IL21, and TGF-β, as well as other important factors that promote differentiation, as IL-12, IL-6, IL-2, IL-23, and IL-10.

In some embodiments, the concentration of T cells can be determined by flow cytometry analysis of cells immunostained for CD45, TCR-β, or CD4. In some embodiments, functional activity of T cells is measured by isolating T cells and using the Elispots kit; for example. In some embodiments, T cells are isolated and grown in a medim, the medium is then collected and the level of secreted cytokines is quantified using ELISA. In some embodiments, the ratio of different cytokines, as IL2/IL10, IL2/IL4, and INFγ/TGFβ in the conditioned medium is evaluated.

The terms “immune checkpoint” and “checkpoint” are used herein interchangeably, having all the same qualities and meanings. In some embodiments, the terms “immune response” and “inflammatory response” are used herein interchangeably, having all the shame qualities and meanings.

### Ophthalmic Inflammatory Conditions

In some embodiments, provided herein is a method for treating an ophthalmic inflammatory condition, the method comprising ocular administration of a composition comprising an immune checkpoint inhibitor. A skilled artisan would appreciate that an ophthalmic inflammatory condition is a medical condition in which the immune system attacks the eye and/or its surrounding tissues. While an inflammatory response is normal and healthy after an injury or other stimuli, in ophthalmic inflammatory conditions said response comprises an abnormal and extended inflammation that results in damage to eye tissues, vision impairment, or chronic pain.

In some embodiments, an ophthalmic inflammatory disease comprises corneal edema. In some embodiments, an ophthalmic inflammatory disease comprises Tyndall's effect signs in the anterior chamber. In some embodiments, an ophthalmic inflammatory disease comprises cells in the anterior chamber. In some embodiments, an ophthalmic inflammatory disease comprises iris exudation. In some embodiments, an ophthalmic inflammatory disease comprises vitreous opacity. In some embodiments, an ophthalmic inflammatory disease comprises retinal vascular leakage. In some embodiments, an ophthalmic inflammatory disease comprises increased immune biomarkers in intraocular fluid. In some embodiments, an ophthalmic inflammatory disease comprises increased immune biomarkers in systemic peripheral blood. In some embodiments, the immune biomarkers comprise IFN-γ, IL-2, IL-17, or any combination thereof.

In some embodiments, an ophthalmic inflammatory disease comprises infiltration of immune cells into the retina. In some embodiments, an ophthalmic inflammatory disease comprises T cell infiltration into the retina. In some embodiments, an ophthalmic inflammatory disease comprises T cell infiltration into ganglion cell layer. In some embodiments, an ophthalmic inflammatory disease comprises CD4+ cell infiltration into the retina. In some embodiments, an ophthalmic inflammatory disease does not comprise CD8+ infiltration into the retina. In some embodiments, an ophthalmic inflammatory disease comprises secretion of inflammatory factors in the retina.

In some embodiments, an ophthalmic inflammatory disease comprises interferon-γ (IFN-γ) in the intraocular fluid. In some embodiments, an ophthalmic inflammatory disease comprises interleukin (IL)2 in the intraocular fluid. In some embodiments, an ophthalmic inflammatory disease comprises IL17 the intraocular fluid. In some embodiments, an ophthalmic inflammatory disease does not comprise interleukin IL17, IL4, or transforming growth factor-β (TGF-β) secretion in the retina.

In some embodiments, an ophthalmic inflammatory disease comprises glaucoma. In some embodiments, glaucoma comprises open-angle glaucoma. In some embodiments, glaucoma comprises angle-closure glaucoma. In some embodiments, glaucoma comprises normal-tension glaucoma. In some embodiments, glaucoma comprises congenital glaucoma. In some embodiments, glaucoma comprises primary glaucoma. In some embodiments, glaucoma comprises secondary glaucoma. In some embodiments, glaucoma comprises pigmentary glaucoma. In some embodiments, glaucoma comprises pseudoexfoliative glaucoma.

In some embodiments, glaucoma comprises traumatic glaucoma. In some embodiments, glaucoma comprises neovascular glaucoma. In some embodiments, glaucoma comprises Irido Corneal Endothelial Syndrome (ICE). In some embodiments, glaucoma comprises uveitic glaucoma. In some embodiments, glaucoma comprises open-angle glaucoma.

In some embodiments, an ophthalmic inflammatory disease comprises uveitis. In some embodiments, an ophthalmic inflammatory disease comprises age-related macular degeneration (AMD). In some embodiments, an ophthalmic inflammatory disease comprises diabetic retinopathy. In some embodiments, an ophthalmic inflammatory disease comprises proliferative vitreoretinopathy. In some embodiments, an ophthalmic inflammatory disease comprises acute optic nerve ischemia.

In some embodiments, an ophthalmic inflammatory disease comprises keratitis. In some embodiments, an ophthalmic inflammatory disease comprises scleritis. In some embodiments, an ophthalmic inflammatory disease comprises optic neuritis. In some embodiments, an ophthalmic inflammatory disease comprises optic neuromyelitis. In some embodiments, an ophthalmic inflammatory disease comprises endophthalmitis.

In some embodiments, an ophthalmic inflammatory disease comprises sputum cellulitis. In some embodiments, an ophthalmic inflammatory disease comprises retinitis pigmentosa. In some embodiments, an ophthalmic inflammatory disease comprises central retinal vein occlusion. In some embodiments, an ophthalmic inflammatory disease comprises central retinal artery occlusion. In some embodiments, an ophthalmic inflammatory disease comprises anterior ischemic optic neuropathy.

In some embodiments, an ophthalmic inflammatory disease comprises thyroid associated ophthalmopathy. In some embodiments, an ophthalmic inflammatory disease comprises optic nerve maternal tumor. In some embodiments, an ophthalmic inflammatory disease comprises choroidal melanoma. In some embodiments, an ophthalmic inflammatory comprises a number of ophthalmic conditions.

### Methods of Treating Ophthalmic Inflammatory Conditions

In some embodiments, provided herein is a method for treating an ophthalmic inflammatory condition in a subject, by administering an immune checkpoint inhibitor. The terms “treating” and “treatment” as used herein refer to the administration of an immune checkpoint inhibitor to a clinically symptomatic individual afflicted with an ophthalmic or an ophthalmic inflammatory condition, disorder, or disease, so as to effect a reduction in the severity and/or the frequency of the clinical symptoms of the condition, disorder, or disease. In some embodiments, a reduction in the severity and/or the frequency of the clinical symptoms of the condition, disorder, or disease, are referred herein as a “desired therapeutic effect”.

In some embodiments, disclosed herein is a composition comprising an immune checkpoint inhibitor for use in treating an ophthalmic inflammatory condition by ocular administration. In some embodiments, disclosed herein is a composition comprising a combination of at least two immune checkpoint inhibitors for use in treating an ophthalmic inflammatory condition by ocular administration. In some embodiments, disclosed herein is a composition disclosing a combination of any of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR) for use in treating an ophthalmic inflammatory condition by ocular administration.

In some embodiments, treating an ophthalmic immune condition comprises eliminating the symptoms of the condition. In some embodiments, treating an ophthalmic immune condition comprises eliminating the underlying cause of the condition. In some embodiments, treating an ophthalmic immune condition comprises facilitating improvement or curing the damage caused by the condition.

In some embodiments, treating an ophthalmic inflammatory condition comprises ameliorating any symptom or symptoms associated with said condition. In some embodiments, treating an ophthalmic condition comprises improving peripheral vision. In some embodiments, treating an ophthalmic inflammatory condition comprises decreasing the optic nerve cupping. In some embodiments, treating an ophthalmic inflammatory condition comprises broadening the nerve fiber layer.

In some embodiments, treating an ophthalmic inflammatory condition comprises diminishing unilateral eye pain. In some embodiments, treating an ophthalmic inflammatory condition comprises improving a vision parameter. In some embodiments, treating an ophthalmic inflammatory condition comprises diminishing nausea and vomiting. In some embodiments, treating an ophthalmic inflammatory condition comprises diminishing red eye.

In some embodiments, treating an ophthalmic inflammatory condition comprises diminishing swollen eye. In some embodiments, treating an ophthalmic inflammatory condition comprises diminishing eye enlargement. In some embodiments, treating an ophthalmic inflammatory condition comprises diminishing light sensitivity. In some embodiments, treating an ophthalmic inflammatory condition comprises diminishing tearing.

In some embodiments, treating an ophthalmic inflammatory condition comprises restraining inflammation. In some embodiments, treating an ophthalmic inflammatory condition comprises decreasing the concentration of inflammatory cells in the eye. In some embodiments, treating an ophthalmic inflammatory condition comprises decreasing the concentration of inflammatory cytokines in the eye. In some embodiments, treating an ophthalmic inflammatory condition comprises improving any combination of the symptoms thereof.

In some embodiments, treating an ophthalmic inflammatory condition comprises preventing loss of peripheral vision. In some embodiments, treating an ophthalmic inflammatory condition comprises preventing thinning of the nerve fiber layer. In some embodiments, treating an ophthalmic inflammatory condition comprises preventing severe eye pain. In some embodiments, treating an ophthalmic inflammatory condition comprises preventing cloudy vision. In some embodiments, treating an ophthalmic inflammatory condition comprises preventing red eye, swollen eye, eye enlargement, light sensitivity, or tearing.

In some embodiments, treating an ophthalmic inflammatory condition comprises reducing the concentration of immune cells in a body fluid. In some embodiments, treating an ophthalmic inflammatory condition comprises reducing the concentration of immune factors in a body fluid.

In some embodiments, an immune factor comprises a pro-inflammatory cytokine. In some embodiments, an immune factor comprises IFN-γ, IL-17, IL21, TGF-β, IL-12, IL-6, IL-2, IL-23, or IL-10, or any combination thereof.

In some embodiments, provided herein is a method for preventing an ophthalmic inflammatory condition, the method comprising ocular administration of an immune checkpoint inhibitor to a clinically asymptomatic individual with predisposition to develop an ophthalmic or an ophthalmic inflammatory condition, disorder, or disease, so as to prevent or reduce the severity and/or the frequency of the future clinical symptoms of the condition, disorder, or disease. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause.

In some embodiments, provided herein is a method for treating an ophthalmic inflammatory condition, the method comprising intraocular administration of a composition comprising a CD27 inhibitor. In some embodiments, provided herein is a method for treating an ophthalmic inflammatory condition, the method comprising intraocular administration of a composition comprising a CD28 inhibitor. In some embodiments, provided herein is a method for treating an ophthalmic inflammatory condition, the method comprising intraocular administration of a composition comprising a CD40 inhibitor.

In some embodiments, provided herein is a method for treating an ophthalmic inflammatory condition, the method comprising intraocular administration of a composition comprising a CD48 inhibitor. In some embodiments, provided herein is a method for treating an ophthalmic inflammatory condition, the method comprising intraocular administration of a composition comprising a CD70 inhibitor. In some embodiments, provided herein is a method for treating an ophthalmic inflammatory condition, the method comprising intraocular administration of a composition comprising a CD80 inhibitor.

In some embodiments, provided herein is a method for treating an ophthalmic inflammatory condition, the method comprising intraocular administration of a composition comprising a CD86 inhibitor. In some embodiments, provided herein is a method for treating an ophthalmic inflammatory condition, the method comprising intraocular administration of a composition comprising a CD122 inhibitor. In some embodiments, provided herein is a method for treating an ophthalmic inflammatory condition, the method comprising intraocular administration of a composition comprising a CD134 inhibitor.

In some embodiments, provided herein is a method for treating an ophthalmic inflammatory condition, the method comprising intraocular administration of a composition comprising a CD137 inhibitor. In some embodiments, provided herein is a method for treating an ophthalmic inflammatory condition, the method comprising intraocular administration of a composition comprising a CD137L inhibitor. In some embodiments, provided herein is a method for treating an ophthalmic inflammatory condition, the method comprising intraocular administration of a composition comprising a CD152 inhibitor.

In some embodiments, provided herein is a method for treating an ophthalmic inflammatory condition, the method comprising intraocular administration of a composition comprising a CD154 inhibitor. In some embodiments, provided herein is a method for treating an ophthalmic inflammatory condition, the method comprising intraocular administration of a composition comprising a CD244 inhibitor. In some embodiments, provided herein is a method for treating an ophthalmic inflammatory condition, the method comprising intraocular administration of a composition comprising a CD252 inhibitor.

In some embodiments, provided herein is a method for treating an ophthalmic inflammatory condition, the method comprising intraocular administration of a composition comprising a CD255 inhibitor. In some embodiments, provided herein is a method for treating an ophthalmic inflammatory condition, the method comprising intraocular administration of a composition comprising a CD273 inhibitor. In some embodiments, provided herein is a method for treating an ophthalmic inflammatory condition, the method comprising intraocular administration of a composition comprising a CD274 inhibitor.

In some embodiments, provided herein is a method for treating an ophthalmic inflammatory condition, the method comprising intraocular administration of a composition comprising a CD275 inhibitor. In some embodiments, provided herein is a method for treating an ophthalmic inflammatory condition, the method comprising intraocular administration of a composition comprising a CD278 inhibitor. In some embodiments, provided herein is a method for treating an ophthalmic inflammatory condition, the method comprising intraocular administration of a composition comprising a CD357 inhibitor.

In some embodiments, provided herein is a method for treating an ophthalmic inflammatory condition, the method comprising intraocular administration of a composition comprising a GITRL inhibitor. In some embodiments, provided herein is a method for treating an ophthalmic inflammatory condition, the method comprising intraocular administration of a composition comprising a BTN2A1 inhibitor. In some embodiments, provided herein is a method for treating an ophthalmic inflammatory condition, the method comprising intraocular administration of a composition comprising a DC-SIGN inhibitor.

In some embodiments, provided herein is a method for treating an ophthalmic inflammatory condition, the method comprising intraocular administration of a composition comprising a TL1A3 inhibitor. In some embodiments, provided herein is a method for treating an ophthalmic inflammatory condition, the method comprising intraocular administration of a composition comprising a DR3 inhibitor.

In some embodiments, provided herein is a method for treating an ophthalmic inflammatory condition, the method comprising intraocular administration of a composition comprising an adenosine A1 receptor (A1aR) agonist. In some embodiments, provided herein is a method for treating an ophthalmic inflammatory condition, the method comprising intraocular administration of a composition comprising an adenosine A2 receptor (A2aR) agonist. In some embodiments, provided herein is a method for treating an ophthalmic inflammatory condition, the method comprising intraocular administration of a composition comprising an adenosine A3 receptor (A3aR) agonist.

In some embodiments, a subject is a human In some embodiments, a subject is an adult. In some embodiments, a subject is a child. In some embodiments, a subject is an elder. In some embodiments, a subject is a patient suffering from an ophthalmic inflammatory condition. In some embodiments, a subject is an animal. In some embodiments, a subject is a mammal.

### Pharmaceutical Compositions Comprising Immune Checkpoint Inhibitors.

In some embodiments, the herein described immune checkpoint inhibitors are incorporated into “pharmaceutical compositions” suitable for administration. A pharmaceutical composition typically comprises an immune checkpoint inhibitor and a pharmaceutically acceptable carrier. As used herein, the terms “pharmaceutical compositions”, “ophthalmic compositions”, “pharmaceutical formulations”, and “formulations” are interchangeable, having all the same qualities and meanings.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. As used herein, the terms “pharmaceutically acceptable carrier”, “pharmaceutically carrier”, and “ophthalmic carrier” are interchangeable, having all the same qualities and meanings. Suitable carriers are described in the most recent edition of Remington: The Science and Practice of Pharmacy, Nineteenth Ed. (Easton, Pa.: Mack Publishing Company, 1995), a standard reference text in the field, which is incorporated herein by reference.

In some embodiments, the pharmaceutical composition comprises any of the immune checkpoint inhibitors disclosed herein. In some embodiments, the pharmaceutical composition comprises a single immune checkpoint inhibitor. In some embodiments, the pharmaceutical composition comprises two different immune checkpoint inhibitors. In some embodiments, the pharmaceutical composition comprises three different checkpoint inhibitors. In some embodiments, the pharmaceutical composition comprises four immune checkpoint inhibitors. In some embodiments, the pharmaceutical composition comprises a number of immune checkpoint inhibitors.

In some embodiments, the pharmaceutical composition comprises a CD27 inhibitor. In some embodiments, the pharmaceutical composition comprises a CD28 inhibitor. In some embodiments, the pharmaceutical composition comprises a CD40 inhibitor. In some embodiments, the pharmaceutical composition comprises a CD48 inhibitor.

In some embodiments, the pharmaceutical composition comprises a CD70 inhibitor. In some embodiments, the pharmaceutical composition comprises a CD80 inhibitor. In some embodiments, the pharmaceutical composition comprises a CD86 inhibitor. In some embodiments, the pharmaceutical composition comprises a CD122 inhibitor. In some embodiments, the pharmaceutical composition comprises a CD134 inhibitor.

In some embodiments, the pharmaceutical composition comprises a CD137 inhibitor. In some embodiments, the pharmaceutical composition comprises a CD137L inhibitor. In some embodiments, the pharmaceutical composition comprises a CD154 inhibitor. In some embodiments, the pharmaceutical composition comprises a CD244 inhibitor.

In some embodiments, the pharmaceutical composition comprises a CD252 inhibitor. In some embodiments, the pharmaceutical composition comprises a CD255 inhibitor. In some embodiments, the pharmaceutical composition comprises a CD273 inhibitor. In some embodiments, the pharmaceutical composition comprises a CD274 inhibitor.

In some embodiments, the pharmaceutical composition comprises a CD275 inhibitor. In some embodiments, the pharmaceutical composition comprises a CD278 inhibitor. In some embodiments, the pharmaceutical composition comprises a CD357 inhibitor. In some embodiments, the pharmaceutical composition comprises a GITRL inhibitor.

In some embodiments, the pharmaceutical composition comprises a BTN2A1 inhibitor. In some embodiments, the pharmaceutical composition comprises a DC-SIGN inhibitor. In some embodiments, the pharmaceutical composition comprises a TL1A inhibitor. In some embodiments, the pharmaceutical composition comprises a DR3 inhibitor.

In some embodiments, the pharmaceutical composition comprises any combination of an inhibitor of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, and DR3, and an agonist of adenosine A1 receptor (A1aR), adenosine A2 receptor (A2aR), and adenosine A3 receptor (A3aR).

In some embodiments, the pharmaceutical composition comprises an adenosine A1 receptor (A1aR) agonist. In some embodiments, the pharmaceutical composition comprises an adenosine A2 receptor (A2aR) agonist. In some embodiments, the pharmaceutical composition comprises an adenosine A3 receptor (A3aR) agonist.

Checkpoint inhibitors, or pharmaceutical compositions comprising thereof, can be administered to the eye in different manners, depending on the precise nature of the formulation and the desired outcome of the administration. In some embodiments, pharmaceutical compositions are delivered directly to the eye, for example by topical ocular drops or ointments, by slow release devices such as pharmaceutical drug delivery sponges implanted in the cul-de-sac or implanted adjacent to the sclera or within the eye, or by periocular, conjunctival, sub-tenons, intracameral, intravitreal, or intracanalicular injections. In some embodiments, methods of administration disclosed herein deliver pharmaceutical compositions disclosed herein directly to the eye, for example by topical ocular drops or ointments, by slow release devices such as pharmaceutical drug delivery sponges implanted in the cul-de-sac or implanted adjacent to the sclera or within the eye, or by periocular, conjunctival, sub-tenons, intracameral, intravitreal, or intracanalicular injections.

In some embodiments, the pharmaceutical compositions are administered systemically, for example by intravenous, subcutaneous, or intramuscular injections, parenteral, oral, dermal, rectal, or nasal delivery. In some embodiments, methods of administration disclosed herein deliver pharmaceutical compositions disclosed herein systemically, for example by intravenous, subcutaneous, or intramuscular injections, parenteral, oral, dermal, rectal, or nasal delivery.

The pharmaceutical compositions can be administered in any form suitable for ocular drug administration, e.g., dosage forms suitable for topical administration, a solution or suspension for administration as eye drops or eye washes, ointment, gel, cream, liposomal dispersion, colloidal microparticle suspension, or the like. The pharmaceutical compositions can be administered also in an ocular insert, e.g., in an optionally biodegradable controlled release polymeric matrix. The ocular insert is implanted in the conjunctiva, sclera, pars plana, anterior segment, or posterior segment of the eye Implants provide for controlled release of the formulation to the ocular surface, typically sustained release over an extended time period. In some embodiments, the formulation is entirely composed of components that are naturally occurring Generally Regarded as Safe (“GRAS”).

The pharmaceutically acceptable carrier may comprise a wide variety of non-active ingredients which are useful for formulation purposes and which do not materially affect the novel and useful properties of the checkpoint inhibitors disclosed herein. By a “pharmaceutically acceptable” or “ophthalmically acceptable” component is meant a component that is not biologically or otherwise undesirable, i.e., the component may be incorporated into an ophthalmic formulation of checkpoint inhibitors and administered topically to a patient's eye without causing any undesirable biological effects or interacting in a deleterious manner with any of the other components of the formulation composition in which it is contained. When the term “pharmaceutically acceptable carrier” is used to refer to a component other than a pharmacologically active agent, it is implied that the component has met the required standards of toxicological and manufacturing testing or that it is included on the Inactive Ingredient Guide prepared by the U.S. Food and Drug Administration.

The pharmaceutical compositions disclosed herein optionally include various other ingredients, including but not limited to surfactants, tonicity agents, buffers, preservatives, co-solvents and viscosity building agents. Carriers that are at least partially aqueous can comprise thickeners, isotonic agents, buffering agents, and preservatives, providing that any such excipients do not interact in an adverse manner with any of the formulation's other components.

Suitable thickeners will be known to those of ordinary skill in the art of ophthalmic formulation, and include, by way of example, cellulosic polymers such as methylcellulose (MC), hydroxyethylcellulose (HEC), hydroxypropylcellulose (HPC), hydroxypropylmethylcellulose (HPMC), and sodium carboxymethylcellulose (NaCMC), and other swellable hydrophilic polymers such as polyvinyl alcohol (PVA), hyaluronic acid or a salt thereof (e.g., sodium hyaluronate), and crosslinked acrylic acid polymers commonly referred to as “carbomers” (and available from B.F. Goodrich as Carbopol® polymers). In some embodiments, the amount of any thickener is such that a viscosity in the range of about 15 cps to 25 cps is provided, as a solution having a viscosity in the aforementioned range is generally considered optimal for both comfort and retention of the formulation in the eye.

Any suitable isotonic agents and buffering agents commonly used in ophthalmic formulations may be used, providing that the osmotic pressure of the solution does not deviate from that of lachrymal fluid by more than 2-3% and that the pH of the formulation is maintained in the range of about 6.5 to about 8.0, preferably in the range of about 6.8 to about 7.8, and optimally at a pH of about 7.4. Preferred buffering agents include carbonates such as sodium and potassium bicarbonate.

In some embodiments, tonicity agents can be employed to adjust the tonicity of the composition, preferably to that of natural tears for ophthalmic compositions. For example, sodium chloride, potassium chloride, magnesium chloride, calcium chloride, dextrose and/or mannitol are added to the composition to approximate physiological tonicity. Such an amount of tonicity agent varies depending on the particular agent to be added. In some embodiments, the pharmaceutical compositions have a tonicity agent in an amount sufficient to cause the final composition to have an ophthalmically acceptable osmolality of about 150-450 mOsm, or of about 250-350 mOsm.

The pharmaceutically ophthalmic carrier used with the formulations may be of a wide range of types known to those of skill in the art. For example, the formulations are optionally provided as an ophthalmic solution or suspension, in which case the carrier is at least partially aqueous. Optionally, the formulations are ointments, in which case the pharmaceutically acceptable carrier comprises an ointment base. Preferred ointment bases herein have a melting or softening point close to body temperature, and any ointment bases commonly used in ophthalmic preparations are advantageously employed. Common ointment bases include petrolatum and mixtures of petrolatum and mineral oil.

The formulations are optionally prepared as a hydrogel, dispersion, or colloidal suspension. Hydrogels are formed by incorporation of a swellable, gel-forming polymer such as those set forth above as suitable thickening agents (i.e., MC, HEC, HPC, HPMC, NaCMC, PVA, or hyaluronic acid or a salt thereof, e.g., sodium hyaluronate), except that a formulation referred to in the art as a “hydrogel” typically has a higher viscosity than a formulation referred to as a “thickened” solution or suspension. In contrast to such preformed hydrogels, a formulation may also be prepared so as to form a hydrogel in situ following application to the eye. Such gels are liquid at room temperature but gel at higher temperatures (and thus are termed “thermoreversible” hydrogels), such as when placed in contact with body fluids. Biocompatible polymers that impart this property include acrylic acid polymers and copolymers, N-isopropylacrylamide derivatives, and ABA block copolymers of ethylene oxide and propylene oxide (conventionally referred to as “poloxamers” and available under the Pluronic® tradename from BASF-Wyandotte). The formulations can also be prepared in the form of a dispersion or colloidal suspension. Preferred dispersions are liposomal, in which case the formulation is enclosed within “liposomes,” microscopic vesicles composed of alternating aqueous compartments and lipid bilayers. Colloidal suspensions are generally formed from microparticles, i.e., from microspheres, nanospheres, microcapsules, or nanocapsules, wherein microspheres and nanospheres are generally monolithic particles of a polymer matrix in which the formulation is trapped, adsorbed, or otherwise contained, while with microcapsules and nanocapsules, the formulation is actually encapsulated. The upper limit for the size for these microparticles is about 5 mm to about 10 μm.

In some embodiments, the pharmaceutical compositions are incorporated into a sterile ocular insert that provides controlled release of the formulation over an extended time period. In some embodiments, the time period ranges from about 12 hours to 60 days, and possibly up to 12 months or more, following implantation of the insert into the conjunctiva, sclera, or pars plana, or into the anterior segment or posterior segment of the eye. One type of ocular insert is an implant in the form of a monolithic polymer matrix that gradually releases the formulation to the eye through diffusion and/or matrix degradation. In some embodiments, the polymer of the insert is completely soluble and or biodegradable, so that removal of the insert is unnecessary. These types of inserts are well known in the art, and can be composed of a water-swellable, gel-forming polymer such as collagen, polyvinyl alcohol, or a cellulosic polymer.

In some embodiments, the insert that is used to deliver the present formulation comprises a diffusional implant in which the checkpoint inhibitors are contained in a central reservoir enclosed within a permeable polymer membrane that allows for gradual diffusion of the inhibitors out of the implant. In some embodiments, osmotic inserts are used. Osmotic inserts are implants in which the formulation is released as a result of an increase in osmotic pressure within the implant following application to the eye and subsequent absorption of lachrymal fluid.

A skilled artisan will appreciate that the term “controlled release” refers to an agent-containing formulation or fraction thereof in which release of the agent is not immediate. The term is used interchangeably with “nonimmediate release” as defined in Remington: The Science and Practice of Pharmacy, Nineteenth Ed. (Easton, Pa.: Mack Publishing Company, 1995). In some embodiments, the term “controlled release” as used herein refers to “sustained release” rather than to “delayed release” formulations. In some embodiments, the term “sustained release” is used in its conventional sense to refer to a formulation that provides for gradual release of an agent over an extended period of time.

In some embodiments, the ophthalmic formulations are administered topically. Optionally, topical ophthalmic products are packaged in multidose form. Preservatives may thus be required to prevent microbial contamination during use. Suitable preservatives include: chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions will be sterile, but typically unpreserved. Such compositions, therefore, generally will not contain preservatives. However, the ophthalmic compositions are preferably preservative free and packaged in unit dose form.

As used herein “ocular administration” refers to any method for locally administering a drug to the eye. A skilled artisan will appreciate that the most appropriate method of administration depends on the area of the eye to be medicated. The conjunctiva, cornea, anterior chamber, and iris usually respond well to topical therapy. The eyelids can be treated with topical therapy but more frequently require systemic therapy. The posterior segment usually requires systemic therapy, because most topical medications do not penetrate to the posterior segment. Retrobulbar and orbital tissues are treated systemically.

Subconjunctival or sub-Tenon's may increase both drug absorption and contact time. Medications both leak onto the cornea from the entry hole of injection and diffuse through the sclera into the globe. Drugs with low solubility such as corticosteroids may provide a repository of drug lasting days to weeks. For sub-Tenon's injections, about 0.5 mL per site is usually safe and effective.

Retrobulbar medications are used infrequently for therapeutics. Retrobulbar tissues can be anesthetized with local anesthetic. Whenever any medication is placed into the orbit, extreme care must be taken to ensure that the medication is not inadvertently injected into a blood vessel, the optic nerve, or one of the orbital foramen.

In some embodiments, systemic medication is required for posterior segment therapy and to complement topical therapy for the anterior segment. The blood-ocular barriers can limit absorption of less lipophilic drugs, but inflammation will initially allow greater drug concentrations to reach the site. As the eye starts to heal, these barriers will again become effective and can limit further drug penetration.

In some embodiments ocular administration comprises subconjunctival administration. In some embodiments ocular administration comprises intravitreal administration. In some embodiments ocular administration comprises retrobulbar administration. In some embodiments ocular administration comprises intracameral administration. In some embodiments ocular administration comprises a combination of any of the administration routes thereof.

In some embodiments, immune checkpoint inhibitors contact ocular tissues or compartments comprising, but not limited to, the cornea, aqueous humor, iris, sclera, and retina. The term “adnexal” is defined in general terms as the appendages of an organ. In the case of the eye, adnexal defines a number of tissues or surfaces that are in immediate contact with the ocular surface but are not, by definition, comprised by the ocular surface. Exemplary adnexal tissues include, but are not limited to, the eyelids, lacrimal glands, and extraocular muscles. In some embodiments, the immune checkpoint inhibitors contact eyelid structures comprising skin, subcutaneous tissue, orbicularis oculi, orbital septum, tarsal plates, palpebral conjuntiva, and meibomian glands. The adnexal tissues comprise all subdivisions of the lacrimal glands, including the orbital and palpebral portions, as well as all tissues contacted by these glands. Extraocular muscles belonging to this category of adnexal tissues include, but are not limited to, the superior and inferior rectus, lateral and medial rectus, and superior and inferior oblique muscles.

In some embodiments, disclosed herein is a method for treating an ophthalmic inflammatory condition in a subject, said method comprising systemic administration of a composition comprising an immune checkpoint inhibitor.

The dosage used for the pharmaceutical compositions will vary, according to the effective amounts needed to eliminate or improve the ophthalmic inflammatory conditions, or the effective amounts needed to prevent them. In some embodiments, about 0.01 μg, about 0.02 μg, about 0.03 μg, about 0.04 μg, about 0.05 μg, about 0.06 μg, about 0.07 μg, about 0.08 μg, about 0.09 μg, about 0.1 μg, about 0.5 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 20 μg, about 30 μg, about 40 μg, about 50 μg, about 60 μg, about 70 μg, about 80 μg, about 90 μg, or about 100 μg of immune checkpoint inhibitors are delivered per administration. In some embodiments, less than 0.01 μg of immune checkpoint inhibitors are delivered per administration. In some embodiments, more than 100 μg of immune checkpoint inhibitors are delivered per administration.

In some embodiments, the immune checkpoint inhibitor is administered at a dosage range of about 0.01-0.05 μg per administration. In some embodiments, the immune checkpoint inhibitor is administered at a dosage range of about 0.05-0.5 μg per administration. In some embodiments, the immune checkpoint inhibitor is administered at a dosage range of about 0.5-5 μg per administration. In some embodiments, the immune checkpoint inhibitor is administered at a dosage range of about 5-50 μg per administration. In some embodiments, the immune checkpoint inhibitor is administered at a dosage range of about 50-100 μg per administration.

In some embodiments, immune checkpoint inhibitors are administered at a concentration of about 0.01 μg/ml, about 0.05 μg/ml, about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μg/ml, about 0.9 μg/ml, about 1 μg/ml, about 2 μg/ml, about 3 μg/ml, about 4 μg/ml, or about 5 μg/ml. In some embodiments, immune checkpoint inhibitors are administered at a concentration lower than 0.01 μg/ml. In some embodiments, immune checkpoint inhibitors are administered at a concentration higher than 5 μg/ml.

When administered as a intravitreal injection, in some embodiments, about 0.1 ml, about 0.5 ml, about 1 ml, about 2 ml, about 3 ml, about 4 ml, about 5 ml, about 6 ml, about 7 ml, about 8 ml, about 9 ml, about 10 ml, about 25 ml, or about 50 ml of the ophthalmic composition are injected into the eye. In some embodiments, less than 0.1 ml of the ophthalmic composition is injected into the eye. In some embodiments, more than 50 ml of the ophthalmic composition are injected into the eye.

In some embodiments, the pharmaceutical composition is administered once. In some embodiments, the pharmaceutical composition is administered twice. In some embodiments, the pharmaceutical composition is administered three times. In some embodiments, the pharmaceutical composition is administered four times. In some embodiments, the pharmaceutical composition is administered six times. In some embodiments, the pharmaceutical composition is administered seven times. In some embodiments, the pharmaceutical composition is administered eight times. In some embodiments, the pharmaceutical composition is administered nine times. In some embodiments, the pharmaceutical composition is administered ten times. In some embodiments, the pharmaceutical composition is administered a number of times. In some embodiments, the pharmaceutical composition is administered a number of times until achieving a therapeutic effect.

In some embodiments, the pharmaceutical composition is administered periodically. In some embodiments, the pharmaceutical composition is administered daily. In some embodiments, the pharmaceutical composition is administered about once a week. In some embodiments, the pharmaceutical composition is administered twice a week. In some embodiments, the pharmaceutical composition is administered about once a month. In some embodiments, the pharmaceutical composition is administered about every 3 months. In some embodiments, the pharmaceutical composition is administered about twice a year. In some embodiments, the pharmaceutical composition is administered about once a year.

### EXAMPLES

### Example 1

### General Methods

Glaucoma mouse model. Two glaucoma mice models were used in the following experiments. In the first model, high intraocular pressure was induced in adult C57BL/6J mice by injecting polystyrene microparticles. First, mice were anesthetized by intraperitoneal injection of a mixture of ketamine (120 mg/kg) and xylazine (12 mg/kg). Then, a small puncture in the center of the right cornea of the mouse was incised with a 30 G needle (BD, USA). Then, 3 to 4 ml of polystyrene particles of 15 μm diameter (Invitrogen, Oregon, USA) were injected at a 5×10⁶/ml concentration into the right anterior chamber of the eye by a glass micro-injector. Left eyes were used as controls, by injecting 3 to 4 ml of PBS into the left anterior chamber in a similar manner

In the second glaucoma model, high intraocular pressure was spontaneously generated in transgenic mouse DBA/2J (Secano Biotechnology Co., Ltd.) from the age of 6 months.

Model of acute optic nerve ischemia. Acute ocular ischemia was induced by the method of anterior chamber perfusion. A puncture was performed in the anterior chamber on the lower nasal corneoscleral margin using a 30 G intravenous infusion needle. Once the needle was fixed, saline solution was injected through it. The intraocular pressure rapidly reached 80 mmHg (i.e., 114 cm H2O, 1 mmHg=0.133 kPa), and that pressure was maintained for 1 hour.

Model of uveitis. HS-AgP35 lyophilized powder was dissolved at a 4 mg/mL concentration, mixed with an equal amount of complete freund adjuvat (CFA), and fully emulsified. Lewis mice were anesthetized with chloral hydrate, and then 0.1 ml of the HS-Ag emulsification was subcutaneously injected in each hind foot pad, in each hind leg, and in the back. Further 0.1 mL of DTP vaccine was intraperitoneally injected. Mice received 2 sets of the described immunization at a 1 week interval. 1 day after the second HS-Ag immunizations, mice were injected with 0.5 μL of 450 μg/mL typhoid bacillus endotoxin into the flat vitreous portion of the eye.

Model of diabetic retinopathy. Six weeks old C57BL/6 mice were intraperitoneally injected with STZ (60 mg/kg) for 3 consecutive days. Control mice were intraperitoneally injected with the same volume of PBS buffer. Blood samples were taken from the tail vein one week following the injections. Blood glucose of over 250 mg/dl or 13.9 mM were indicative of successful modeling of diabetes. Mice were then grown for 3 months.

Treatment with immune checkpoint inhibitors. After establishing the respective disease model, mice were injected with 2 μl of either a single checkpoint inhibitor, or a combination of checkpoint inhibitors, as indicated in each example, at a 1 μg/ml concentration, into the vitreous cavity of the eye.

TABLE 1
Immune checkpoint inhibitors used in the experiments.

| Immune | Immune checkpoint | |
|---|---|---|
| checkpoint | inhibitor | Dosage |
| CD28 | Anti-CD28 Antibody | 2 μl per administration, at 1 μg/ml |
| | (Sino Biological, Cat: | concentration. |
| | 11524-R007) | |
| CD86 | Anti-CD86 Antibody | 2 μl per administration, at 1 μg/ml |
| | (Sino Biological, Cat: | concentration. |
| | 10699-RP02) | |
| CD80 | Anti-CD80 Antibody | 2 μl per administration, at 1 μg/ml |
| | (Sino Biological, Cat: | concentration. |
| | 50446-R014) | |
| CD40 | Anti-CD40 Antibody | 2 μl per administration, at 1 μg/ml |
| | (Sino Biological, Cat: | concentration. |
| | 101510-T36) | |
| CD154 | Anti-CD154 Antibody | 2 μl per administration, at 1 μg/ml |
| | (Sino Biological, | concentration. |
| | Cat: 10239-MM04) | |
| CD137 | Anti-CD137 Antibody | 2 μl per administration, at 1 μg/ml |
| | (Sino Biological, | concentration. |
| | Cat: 10041-RP02) | |
| CD137L | Anti-CD137L Antibody | 2 μl per administration, at 1 μg/ml |
| | (Sino Biological, | concentration. |
| | Cat: 50067-RP02) | |
| CD27 | Anti-CD27 Antibody | 2 μl per administration, at 1 μg/ml |
| | (Sino Biological, | concentration. |
| | Cat: 10039-R025-A) | |
| CD70 | Anti-CD70 Antibody | 2 μl per administration, at 1 μg/ml |
| | (Sino Biological, | concentration. |
| | Cat: 101956-T32) | |
| CD122 | Anti-CD122 Antibody | 2 μl per administration, at 1 μg/ml |
| | (ThermoFisher, Cat: 17- | concentration. |
| | 1222-80) | |
| CD48 | Anti-CD48 Antibody | 2 μl per administration, at 1 μg/ml |
| | (Sino biological, Cat: | concentration. |
| | 10797-R248) | |
| CD278 | Anti-CD278 Antibody | 2 μl per administration, at 1 μg/ml |
| | (ThermoFisher, | concentration. |
| | Cat: 14-9949-82) | |
| CD275 | Anti-CD275 Antibody | 2 μl per administration, at 1 μg/ml |
| | (Sino Biological, | concentration. |
| | Cat: 11559-MM01-A) | |
| CD357 | Anti-CD357 Antibody | 2 μl per administration, at 1 μg/ml |
| | (Sino Biological, | concentration. |
| | Cat: 13643-R002) | |
| CD279 | Anti-CD279 Antibody | 2 μl per administration, at 1 μg/ml |
| | (Sino Biological, | concentration. |
| | Cat: 10377-HN94) | |
| CD134 | Anti-CD134 Antibody | 2 μl per administration, at 1 μg/ml |
| | (Sino Biological, | concentration. |
| | Cat: 10481-RP01) | |
| CD255 | Anti-CD255 Antibody | 2 μl per administration, at 1 μg/ml |
| | (Biolegend, | concentration. |
| | Cat: 120005) | |
| CD244 | Anti-CD244 Antibody | 2 μl per administration, at 1 μg/ml |
| | (Sino Biological, | concentration. |
| | Cat: 10042-R025) | |
| A2aR | ADORA2A Antibody | 2 μl per administration, at 1 μg/ml |
| | (Biolegend, | concentration. |
| | Cat: 120005MA5-31611) | |

Pathologic assessments. After mice were sacrificed, optic nerve and retinal patches were taken to assess optic nerve damage. The optic nerve was fixed overnight with Karnovsky solution. Transverse sections of the optic nerve were obtained 2 mm after the eyeball and observed by electron microscopy (EM410, Philips). For retinal patches the eyeball was fixed with 4% paraformaldehyde overnight. Retinal patches and frozen sections were taken. Retinal ganglion cells (RGCs) were labeled with beta 3 tubulin (Invitrogen) and recorded using a confocal laser microscope (Olympus FV1000).

Flow cytometry was performed according to the following protocol. RCG or blood-derived lymphocyte cells were homogenized in PBS supplemented with digestive enzymes. Cells were then incubated with labeled antibodies (CD25, Foxp3, CTLA4, Nrp1, CD73, CD45, IFN-γ, IL-17, IL21, TGF-β, IL-12, IL-6, IL-2, IL-23, and IL-10). For determination of CD45RO+/CD45RA+ cells ratio, first, CD4+ labeled T cells were isolated from peripheral blood, then they were labeled with anti-FOXP3 isolated according to FOXP3 expression, cells were labeled with anti-CD45RO and anti-CD45RA.

RT-PCR detection of transcription factors was performed according to the following protocol. RNA from mouse retina or optic nerve was extracted by centrifugation using the Trizol method. RNA purity was measured and RNA concentration was calculated. cDNA was synthesized using a reverse transcription kit (Invitrogen) and stored at −20° C. Primer sequences of T-bet, RORγt, BCL6, FOXP3, and LAG3 were designed according to Genebank sequences. Fluorescence amplification was performed using SYRB® Premix Ex Taq™ and Light Cycler PCR Amplifier (Roche).

T cell responses were tested by Elispot according to the following protocol. 2 ml of blood samples were collected from the main abdominal vein with a 5 ml needle, and were then placed in an anti-coagulation tube, centrifuged at 2000 r/min for 5 min, and the supernatant was removed for 10 μl. An antigen ELISA kit (invitrogen, USA) was used to detect protein expression, and signals were read by a microplate reader at 410 nm.

### Example 2

### Effect of Immune Checkpoint Inhibitors in a Model of Glaucoma

Objective: Assessing whether treatment with immune checkpoint inhibitors, as anti-CD28, anti-CD86, and anti-CD80 antibodies, ameliorate glaucoma symptoms.

Methods: Glaucoma was induced in adult C57BL/6J mice by injecting polystyrene microparticles into their eyes. Antibodies anti-CD28; anti-CD86; anti-CD80; a combination of anti-CD80 and anti-CD28; a combination of anti-CD86 and anti-CD28; and a combination of anti-CD28, anti-CD86, and anti-CD80 were injected to the vitreous cavity of the mice eyes (5 mice per treatment) immediately after the injection of the microparticles. IgG was injected as control. Retinal ganglion cell injury and optic nerve axonal injury were observed after 8 weeks. A description of the methods can be found in Example 1.

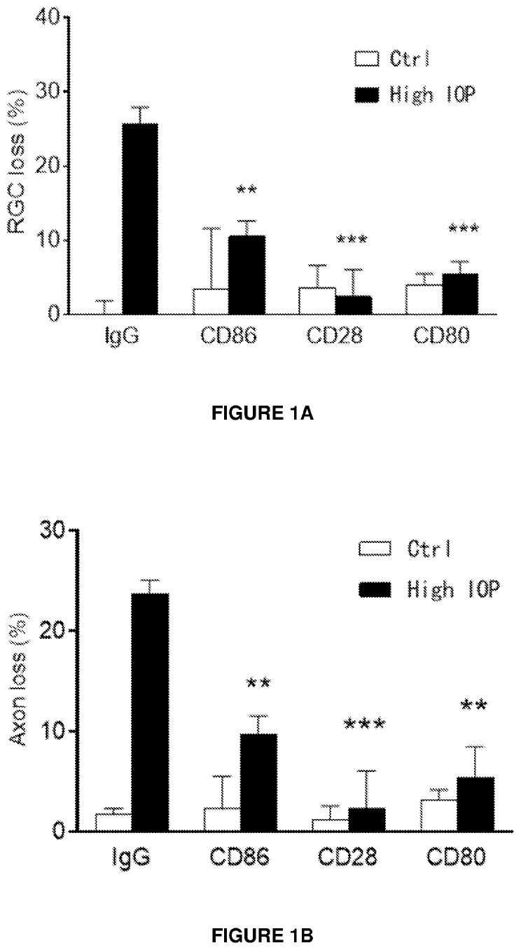

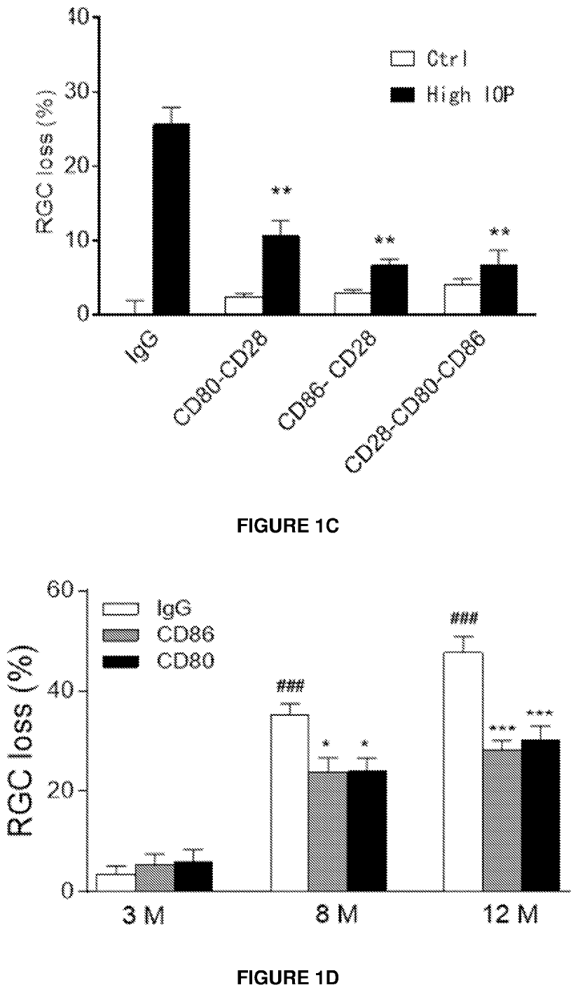

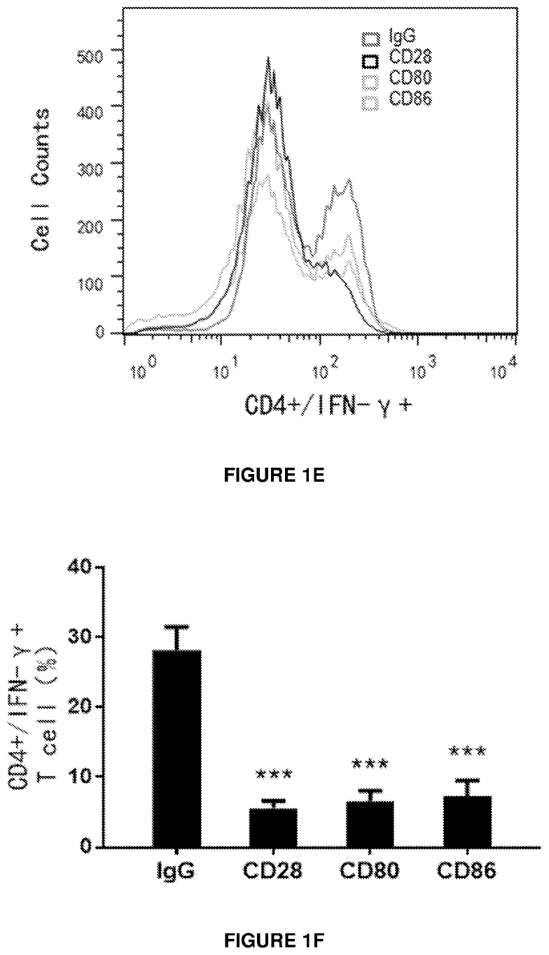

Results: Glaucoma mice injected with anti-CD28, anti-CD86, or anti-CD80 antibodies had decreased RGCs loss compared to IgG injected mice, as revealed by beta 3 tubulin staining (FIG. 1A). Similarly, mice injected with anti-CD28, anti-CD86, or anti-CD80 antibodies had decreased axon loss compared to IgG injected mice, as revealed by Karnovsky staining (FIG. 1B). A combined injection of anti-CD80 and anti-CD28; anti-CD86 and anti-CD28; or anti-CD28, anti-CD86, and anti-CD80 had an enhanced protective effect on RGCs (FIG. 1C).

Conclusions: The present experiment demonstrates that immune checkpoint inhibitors can effectively reduce the persistent damage of glaucoma on RGCs and optic axons. The experiment further demonstrates that checkpoint inhibitors have a synergistic therapeutic effect when combined.

### Example 3

### Validation of Immune Checkpoint Inhibitors Effect in a Second Model of Glaucoma

Objective: Validating that immune checkpoint inhibitors ameliorate glaucoma by using a second mice model of glaucoma.

Methods: At 6 months of age, DBA/2J transgenic mouse with high intraocular pressure were randomly divided into groups of 5 animals, and were administered with anti-CD28, anti-CD86, and anti-CD80 antibodies, or IgG to the vitreous cavity of the eye at intervals of 7-10 days. Retinal ganglion cell (RGC) injury and optic nerve axonal injury, as well as CD4+ T cell ratio was observed. Data of each group of mice at 3 months of age was used as a control. Half of the mice were sacrificed at 8 months of age, and the second half was sacrificed at 12 months of age. A description of the methods can be found in Example 1.

Results: Mice injected with anti-CD28, anti-CD86, or anti-CD80 antibodies had decreased RGCs loss compared to IgG injected mice, both at 8 and 12 months of age, as revealed by beta 3 tubulin staining (FIG. 1D).

Further, the CD4+/IFNγ+ T cell ratio was significant decreased in DBA/2J mice treated with anti-CD28, anti-CD86, and anti-CD80 antibodies, compared with the group injected with IgG at both 8 and 12 months (FIGS. 1E and 1F).

Conclusions: Immune checkpoint inhibitors ameliorate glaucoma symptoms in different mouse models. The reduction of CD4+/IFNγ+ T cell ratio indicates that immune checkpoint inhibitors effectively reduce the inflammatory response associated with glaucoma and regulate abnormal T cell activation, thereby protecting the retina and the optic nerve, and reversing glaucoma damage.

### Example 4

### Immune Checkpoint Inhibitors Inhibit Inflammatory Responses Associated with Glaucoma

Objective: To elucidate the mechanism of action by which immune checkpoint inhibitors ameliorate glaucoma symptoms.

Methods: Glaucoma was induced in adult C57BL/6J mice by microparticles injection. Anti-CD28 or anti-CD86 antibodies or IgG were injected to the vitreous cavity of mice eyes. One week after the injection, peripheral blood and spleen were taken, and cells were analyzed by either flow cytometry or Elispot. The concentration of CD4+, IFNγ+, IL-4+, IL-17+ cells was measured by flow cytometry. To elucidate the ratio between memory T regulatory (mTreg) and primitive Treg cells, the concentration of CD45RO+/FOXP3+ and CD45RA+/FOXP3+ cells was determined. First, peripheral blood was isolated and labeled with an anti-CD4 antibody, CD4+ labeled T cells were then isolated and further labeled with anti-FOXP3 and anti-IL17 antibodies, then FOXP3+ (Treg) cells were isolated and further labeled with anti-CD45RO and anti-CD45RA to assess the concentration of mTreg cells. Concentration of T cells was further measured in spleen by Elispot. A description of the methods can be found in Example 1.

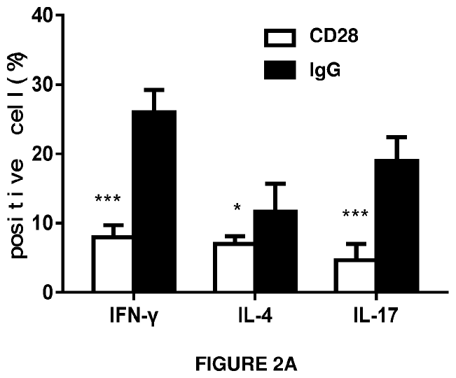

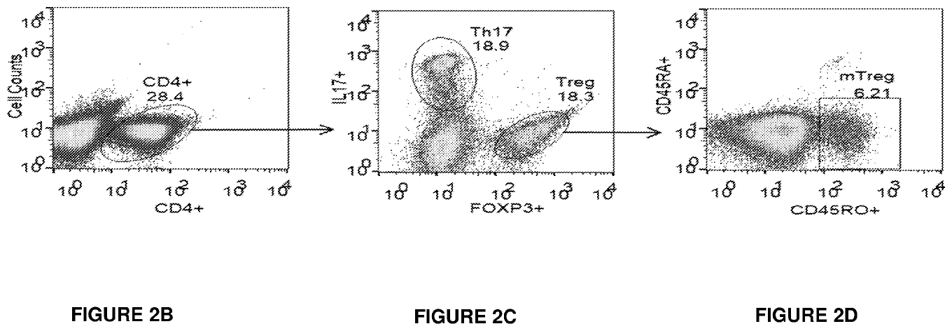

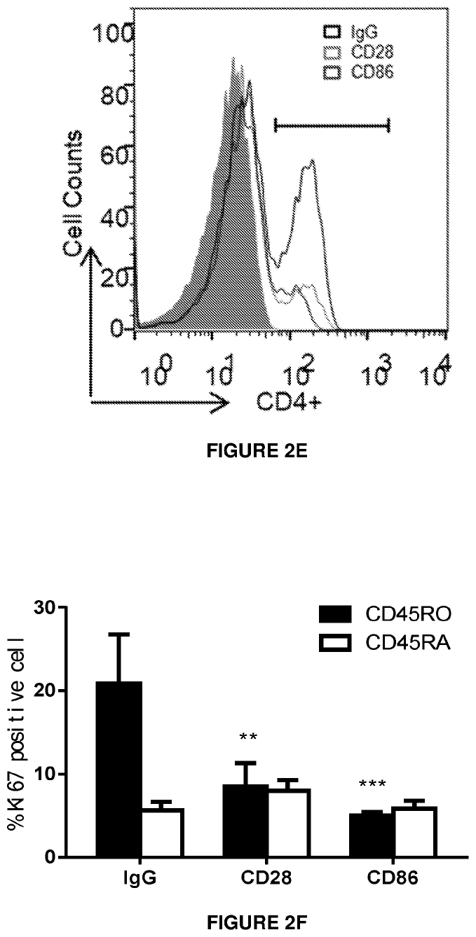

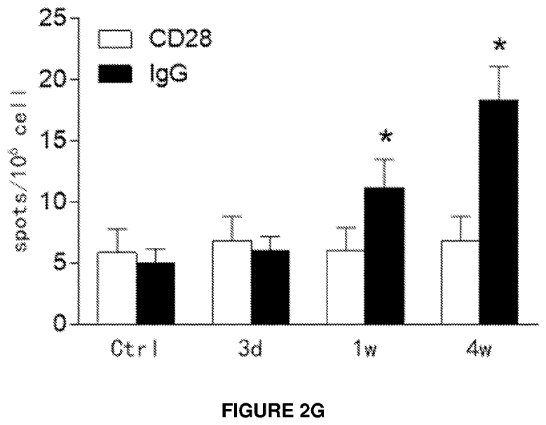

Results: Injection of the anti-CD28 antibody reduced the concentration of IFN-γ, IL-4, and IL-17 positive cells in peripheral blood, indicating a reduction of circulatting Th1, Th2, and Th17 CD4+ T cells (FIG. 2A). To analyze mTreg/primitive Treg ratio, peripheral blood cells were labeled with CD4 (FIG. 2B) and isolated according to its expression. CD4+ cells were then labeled with FOXP3 and IL-17 (FIG. 2C) and isolated according to FOXP3 expression. FOXP3+ isolated cells were labeled with CD45RO and CD45RA (FIG. 2D). Injections of both anti-CD28 and anti-CD86 antibodies decreased peripheral concentration of CD4+ T cells (FIG. 2E), and decreased the ratio of CD45RO+ to CD45RA+ cells (FIG. 2F), indicating decreased concentration of mTreg cells in glaucoma mice treated with immune checkpoint inhibitors. These observation were validated by the decreased concentration of CD4+ T cells in spleen of glaucoma mice 3 days, 1 week, and 4 weeks after treatment with anti-CD28 (FIG. 2G).

Conclusions: The present experiments demonstrate that immune checkpoint inhibitors effectively reduce the T cell mediated inflammatory response associated with glaucoma.

### Example 5

### Effect of Further Immune Checkpoint Inhibitors on Glaucoma

Objective: To test whether further immune checkpoints inhibitors have an ameliorative effect on glaucoma.

Methods: Glaucoma was induced in adult C57BL/6J mice. 2 μl of antibodies targeting CD40, CD154, CD137, CD137L, CD27, CD70, CD122, CD48, CD278, CD275, CD357, CD279, CD134, CD255, or CD244, or IgG, adenosine, or A2aR agonist were administered once a week at a 0.2 μg/ml concentration to the vitreous cavity of the eye. Retinal ganglion cell injury and optic nerve axonal injury was observed after 8 weeks. A description of the methods can be found in Example 1.

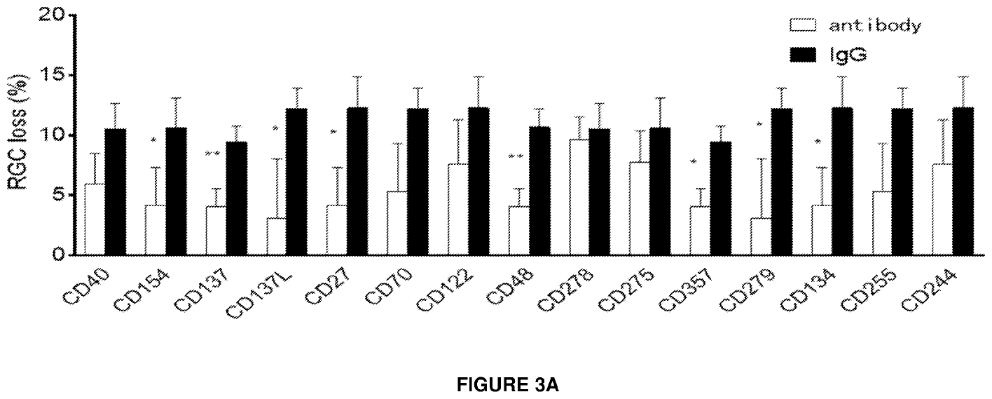

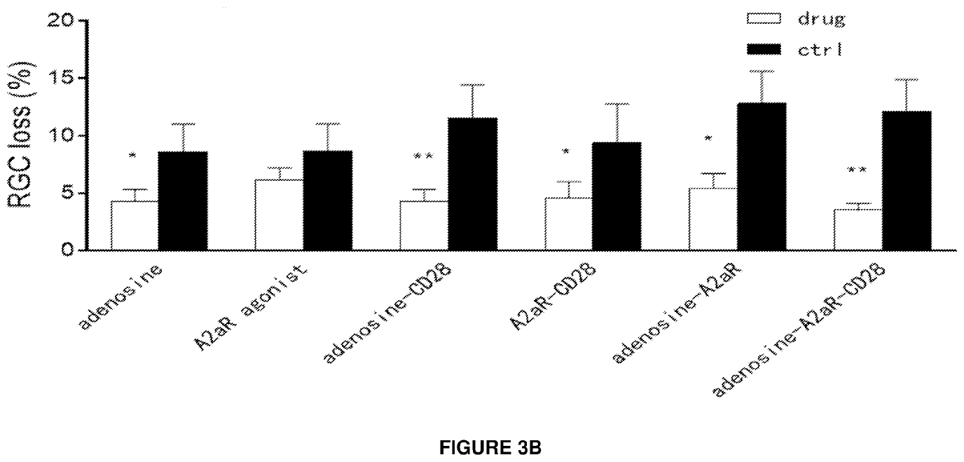

Results: Mice injected with antibodies targeting CD40, CD154, CD137, CD137L, CD27, CD70, CD122, CD48, CD278, CD275, CD357, CD279, CD134, CD255, or CD244 had decreased RGCs loss compared to IgG injected mice, as revealed by beta 3 tubulin staining (FIG. 3A). Similarly, mice injected with adenosine or A2aR had decreased RGCs loss compared to controls (FIG. 3B). Such ameliorative effect was more pronounced when adenosine and A2aR were co-injected with an anti-CD28 antibody (FIG. 3B).

Conclusions: All tested immune checkpoint inhibitors can be used for ameliorating glaucoma symptoms, as RGCs loss.

### Example 6

### Effect of Immune Checkpoint Inhibitors in Acute Optic Nerve Ischemia

Objective: Assessing whether immune checkpoint inhibitors ameliorate acute optic nerve ischemia.

Methods: Acute optic nerve ischemia was induced in adult C57BL/6J mice by the method of anterior chamber perfusion. After 24 hours, anti-CD28, anti-CD86, and anti-CD80, anti-CD27, or anti-CD70 antibody, or IgG were injected to the vitreous cavity of the eye. Mice retinal ganglion cell injury and optic nerve axonal injury was observed after 4 weeks. A description of the methods can be found in Example 1.

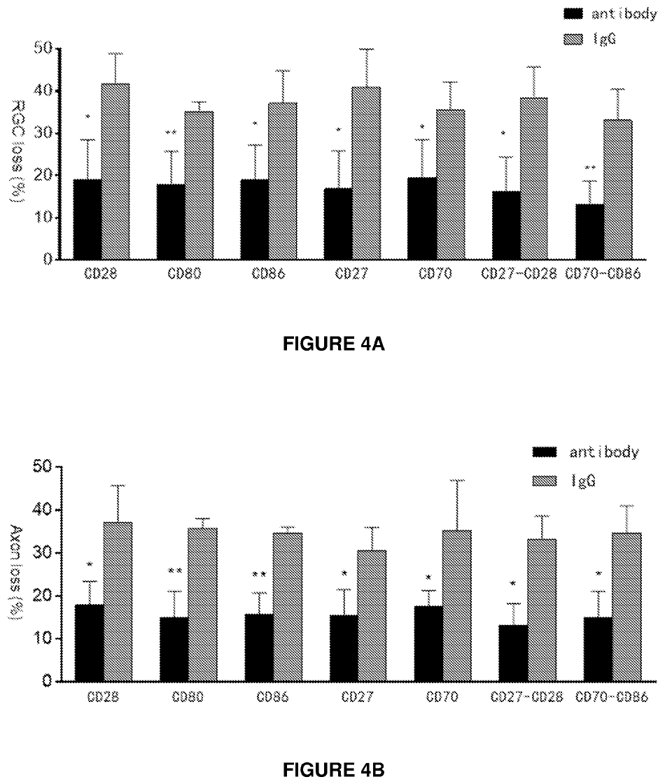

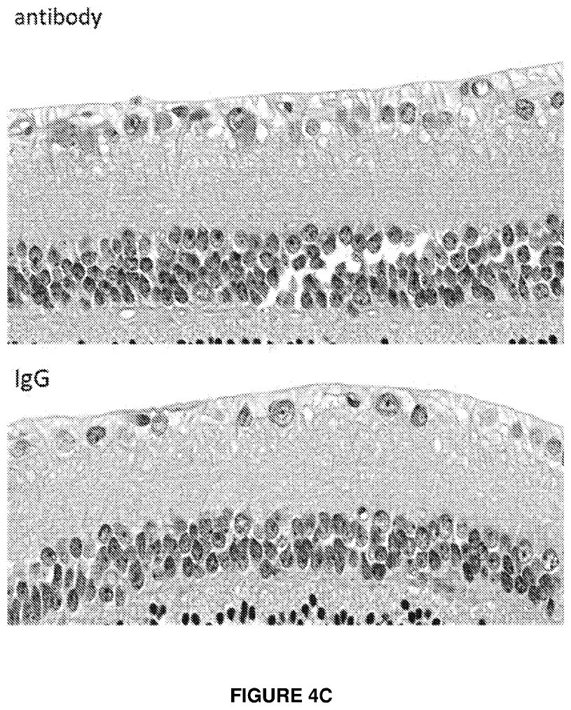

Results: Mice injected with anti-CD28, anti-CD86, anti-CD80, anti-CD27, or anti-CD70 antibodies had decreased RGCs loss compared to IgG injected mice, as revealed by beta 3 tubulin staining (FIG. 4A). Similarly, mice injected with anti-CD28, anti-CD86, anti-CD80, anti-CD27, or anti-CD70 antibodies had decreased axon loss compared to IgG injected mice, as revealed by Karnovsky staining (FIG. 4B). Further, combined injections of anti-CD27 and anti-CD28, or of anti-CD70 and anti-CD86 had an enhanced protective effect on RGCs (FIG. 4A) and axon loss (FIG. 4B).

Conclusions: The present experiment demonstrates that immune checkpoint inhibitors can effectively reduce the optic nerve and retinal damage following acute optic nerve ischemia.

### Example 7

### Effect of Immune Checkpoint Inhibitors in a Uveitis Model

Objective: Assessing whether immune checkpoint inhibitors ameliorate uveitis symptoms.

Methods: Uveitis was induced in adult Lewis mice by immunization with HS-AgP35. Anti-CD28, anti-CD86, anti-CD80, anti-CD278, anti-CD70, anti-CD40, anti-CD154, or anti-CD122 antibody, or IgG were injected to the vitreous cavity of the eye immediately after HS-AgP35 immunization. A clinical score was calculated according to clinical signs of uveitis (Table 2). After 4 weeks, mice RGCs injury and optic nerve axonal injury was observed. RGCs were evaluated by flow cytometry. A description of the methods can be found in Example 1.

TABLE 2
Clinical scores of uveitis.

| Clinical Signs | | Score |
|---|---|---|
| Iris | None | 0 |
| | Mild | 1 |
| | Moderate | 2 |
| | Severe | 3 |
| Pupil | Normal | 0 |
| | Adhesion (synechia) | 1 |
| Anterior chamber | None | 0 |
| exudation | Mild | 1 |
| | Severe | 2 |
| Anterior chamber | None | 0 |
| epyema | Severe | 1 |

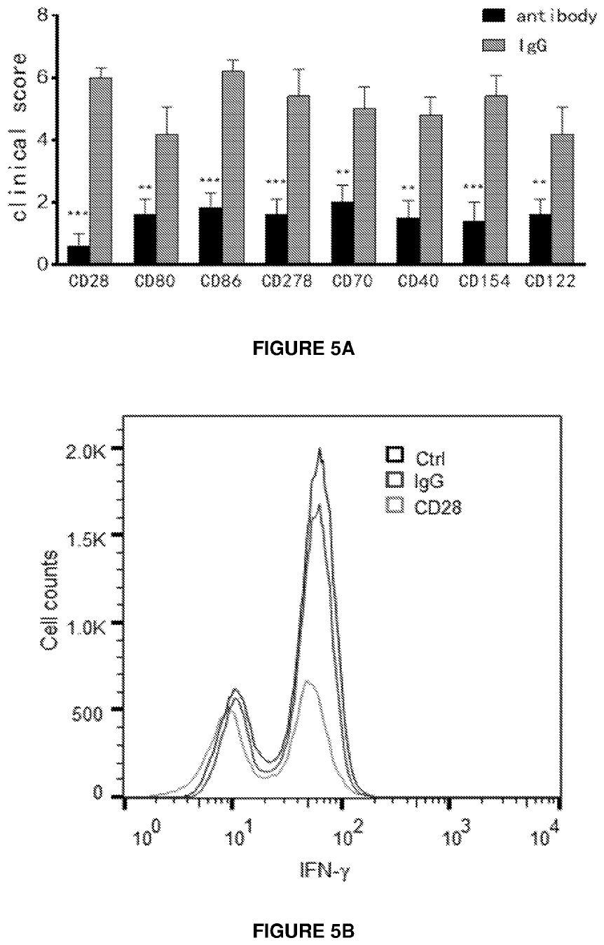

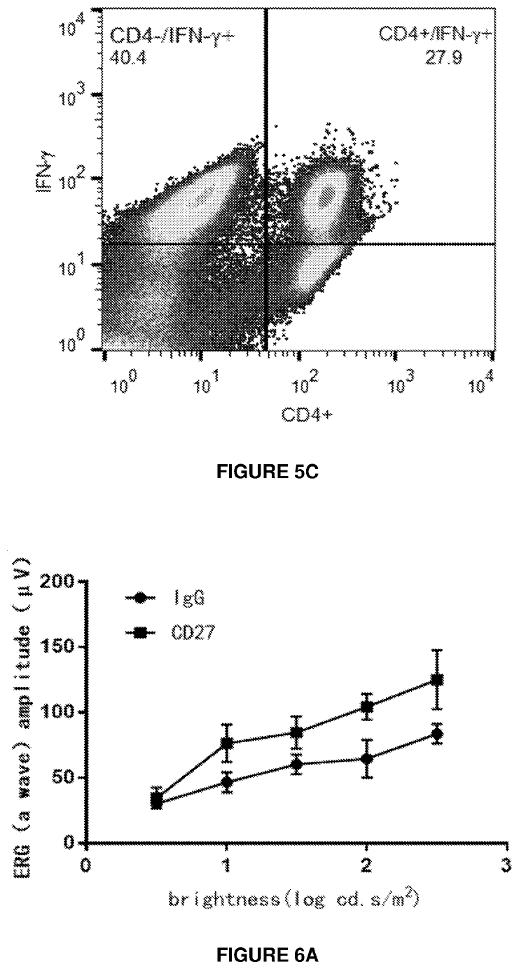

Results: mice injected with anti-CD28, anti-CD86, and anti-CD80, anti-CD278, anti-CD70, anti-CD40, anti-CD154, or anti-CD122 antibodies had lower clinical scores, indicating less clinical signs, than mice injected with IgG (FIG. 5A), indicating that immune checkpoint inhibitors reduced the intraocular inflammatory response associated with uveitis.

Retinal cells from the CD28 antibody injected group and the control group were analyzed by flow cytometry. A large number of inflammatory cells (CD4/IFN-γ positive cells) aggregated in the eye of uveitic mice (FIG. 5B), and a significant increase in inflammatory cell number and inflammatory factors IFN-γ were detected in the glaucoma mice groups and the IgG injected groups. Treatment with anti-CD28 antibody decreased the concentration of IFN-γ secreting T cells (FIGS. 5B and 5C).

Conclusions: The present experiment demonstrates that immune checkpoint inhibitors can effectively reduce uveitis associated symptoms and inflammation.

### Example 8

### Effect of Immune Checkpoint Inhibitors in Diabetic Retinopathy

Objective: Assessing whether immune checkpoint inhibitors ameliorate diabetic retinopathy symptoms.

Methods: Diabetic retinopathy was induced in adult C57BL/6 mice by intraperitoneal injection of STZ. Anti-CD27 or anti-CD28 antibodies were injected to the vitreous cavity of the eye. The antibodies were injected twice a week for 3 months. IgG was a used a control. Visual function was measured by ERG at the end of the 3 months. Mice were then sacrificed, and retinal cells subjected to flow cytometry. A description of the methods can be found in Example 1.

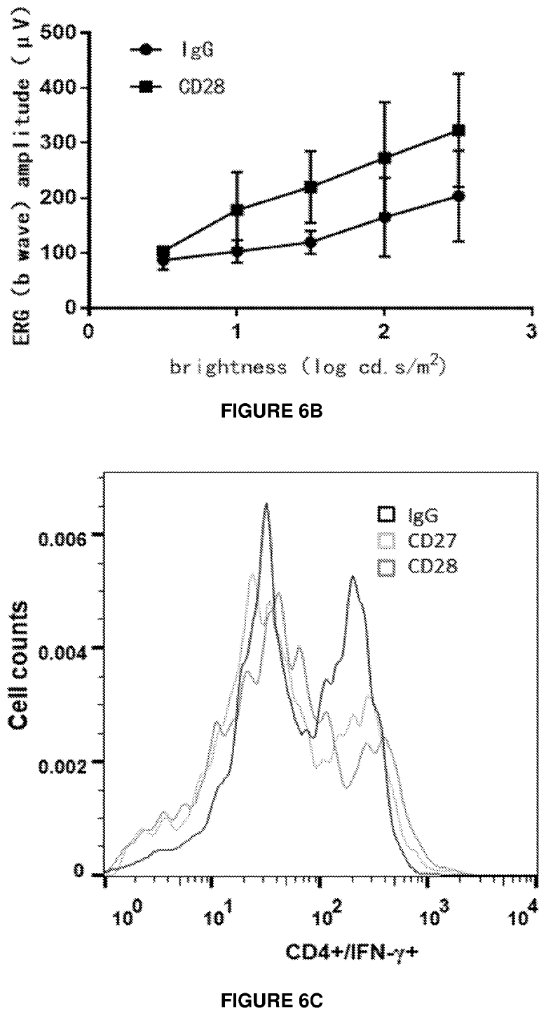

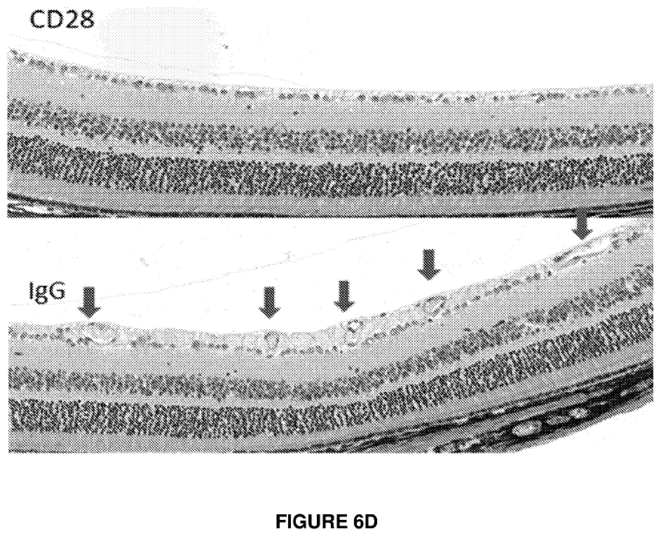

Results: Injection of anti-CD27 antibody or anti-CD28 antibody in diabetic mice significantly increased the ERG a-wave (FIG. 6A) and b-wave (FIG. 6B) amplitude compared to IgG injection, indicating that immune checkpoint inhibitors effectively increase visual function in diabetic retinopathy.

A large number of inflammatory cells aggregated in the eye of diabetic mice. Treatment with anti-CD27 and anti-CD28 antibodies inhibited proliferation of CD4+/IFN-γ+ T cells (FIG. 6C).

The middle and late stages of proliferative diabetic retinopathy are often accompanied by the growth of a large number of neovascular vessels (FIG. 6D, indicated by arrows). Treatment with anti-CD28 antibody growth of neovascular vessels (FIG. 6D).

Conclusions: The present experiment demonstrates that immune checkpoint inhibitors ameliorate visual impairment and other symptoms associated with diabetic retinopathy.

1-18. (canceled)

19. A method for treating an ophthalmic inflammatory condition comprising ocular administration of a composition comprising an immune checkpoint inhibitor that comprises an antagonist of CD27, CD28, CD40, CD48, CD70, CD80, CD86, CD122, CD134, CD137, CD13L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR) in an amount and for a time effective to treat the ophthalmic inflammatory condition.

20. The method of claim 19 wherein the immune checkpoint inhibitor comprises a monoclonal antibody or a binding fragment thereof, a small molecule, or a peptide binding the immune checkpoint.

21. The method of claim 20, wherein the immune checkpoint inhibitor is selected from the group consisting of an anti-CD28 antibody, an anti-CD86 antibody, an anti-CD80 antibody, an anti-CD40 antibody, an anti-CD154 antibody, an anti-CD137 antibody, an anti-CD137L antibody, an anti-CD27 antibody, an anti-CD70 antibody, an anti-CD122 antibody, an anti-CD48 antibody, an anti-CD278 antibody, an anti-CD275 antibody, an anti-CD357 antibody, an anti-CD279 antibody, an anti-CD134 antibody, an anti-CD255 antibody, an anti-CD244 antibody, adenosine, and any combination thereof.

22. The method of claim 19, wherein the composition comprises: 1) a first immune checkpoint inhibitor selected from the group consisting of an antagonist of one or more of CD27, CD28, CD40, CD48, CD70, CD80, CD86, CD122, CD134, CD137, CD137L, CD152, CD154, CD244, CD252, CD255, CD273, CD274, CD275, CD278, CD357, GITRL, BTN2A1, DC-SIGN, TL1A, and DR3; and 2) a second immune checkpoint inhibitor selected from the group consisting of an agonist of one or more of adenosine A1 receptor (A1aR), adenosine A2 receptor (A2aR), and adenosine A3 receptor (A3aR); wherein the combination of the first and the second immune checkpoint inhibitors has a synergistic therapeutic effect upon administration.

23. The method of claim 22, wherein the composition comprises: a) an anti-CD28 antibody and an anti-CD80 antibody; an anti-CD28 antibody and an anti-CD86 antibody; an anti-CD80 antibody and an anti-CD86 antibody; an anti-CD28 antibody and adenosine; an anti-CD28 antibody and an A2aR agonist; an anti-CD27 antibody and an anti-CD28 antibody; an anti-CD70 antibody and an anti-CD85 antibody; an anti-CD27 antibody and an anti-CD279 antibody; an anti-CD27 antibody and an anti-CD273 antibody; an anti-CD27 antibody and an anti-CD274 antibody; an anti-CD28 antibody and an anti-CD279 antibody; an anti-CD28 antibody and an anti-CD273 antibody; an anti-CD28 antibody and an anti-CD274 antibody; an anti-CD80 antibody and an anti-CD279 antibody; an anti-CD80 antibody and an anti-CD273 antibody; an anti-CD80 antibody and an anti-CD274 antibody; an anti-CD86 antibody and an anti-CD279 antibody; an anti-CD86 antibody and an anti-CD273 antibody; or an anti-CD86 antibody and an anti-CD274 antibody; or b) a compound selected from the group consisting of varlilumab, nivolumab, belatacept, ASKP-1240, ISIS 19211, an IL-2/CD40L-expressing leukemia vaccine, 4SCAR19, 4SCAR70, abatacept, acalabrutinib ISIS9133, anti-thymocyte immunoglobulin, denileukin diftitox, AFTVac, GBR 830, BMS-663513, ipilimumab, ASKP-1240, IL-2/CD40L-expressing leukemia vaccine, ruplizumab, AMG 386, JTX-2011, TRX-518, alpha-D-mannose, adenosine, IB-MECA, and combinations thereof.

24. The method of claim 21, wherein the immune checkpoint inhibitor is selected from the group consisting of varlilumab, nivolumab, belatacept, ASKP-1240, ISIS 19211, an IL-2/CD40L-expressing leukemia vaccine, 4SCAR19, 4SCAR70, abatacept, acalabrutinib ISIS9133, anti-thymocyte immunoglobulin, denileukin diftitox, AFTVac, GBR 830, BMS-663513, ipilimumab, ASKP-1240, IL-2/CD40L-expressing leukemia vaccine, ruplizumab, AMG 386, JTX-2011, TRX-518, alpha-D-mannose, adenosine, or IB-MECA, and any combination thereof.

25. The method of claim 19, wherein the ophthalmic inflammatory condition is selected from the group consisting of glaucoma, uveitis, age-related macular degeneration (AMD), diabetic retinopathy, proliferative vitreoretinopathy, acute optic nerve ischemia, keratitis, scleritis, optic neuritis, optic neuromyelitis, endophthalmitis, sputum cellulitis, retinitis pigmentosa, central retinal vein occlusion, central retinal artery occlusion, anterior ischemic optic neuropathy, thyroid associated ophthalmopathy, optic nerve maternal tumor, choroidal melanoma, and combinations thereof.

26. The method of claim 19, wherein the composition is formulated for subconjunctival, intravitreal, retrobulbar, or intracameral administration, or any combination thereof.

27. The method of claim 19, wherein the composition is administered at a dosage of about 0.5 to about 5 μg immune checkpoint inhibitor per administration.

28. The method of claim 19, wherein the composition is formulated to be administered once, or a number of times until achieving a desired therapeutic effect.

29. The method of claim 19, wherein the treating comprises: 1) ameliorating one or more symptoms selected from the group consisting of loss of peripheral vision, optic nerve cupping, thinning of the nerve fiber layer, severe unilateral eye pain, inflammation, cloudy vision, nausea, vomiting, red eye, swollen eye, eye enlargement, light sensitivity, and tearing; or 2) reducing the concentration of immune cells or immune factors in one or more body fluids; or 3) a combination of 1) and 2).

30. A method for treating retinal ganglion inflammation in a mammalian eye, comprising administering to the eye a composition comprising an immune checkpoint inhibitor that comprises an antagonist of CD27, CD28, CD40, CD48, CD70, CD80, CD86, CD122, CD134, CD137, CD13L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR) in an amount and for a time effective to treat the retinal ganglion inflammation in the eye.

31. A method for treating or ameliorating one or more symptoms of glaucoma in a mammalian eye, comprising administering to the eye a composition comprising an immune checkpoint inhibitor that comprises an antagonist of CD27, CD28, CD40, CD48, CD70, CD80, CD86, CD122, CD134, CD137, CD13L, CD152, CD154, CD244, CD252, CD255, CD273, CD274, CD275, CD278, CD357, GITRL, BTN2A1, DC-SIGN, TL1A, or DR3, or an agonist of adenosine A1 receptor (A1aR), adenosine A2 receptor (A2aR), or adenosine A3 receptor (A3aR) in an amount and for a time effective to treat the retinal ganglion inflammation in the eye.
